(19) United States
(12) Patent Application Publication
(10) Pub. No.: US 2009/0209527 A1
(43) Pub. Date: Aug. 20, 2009

(54) Methods for Using Rapamycin Analogues in the Treatment of Neurological Disorders

(75) Inventors: Kevin Pong, West Chester, PA (US); Margaret Maria Zaleska, Narberth, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/388,603

(22) Filed: Feb. 19, 2009

Related U.S. Application Data
(60) Provisional application No. 61/029,783, filed on Feb. 19, 2008.

(51) Int. Cl. A61K 31/5365; A61K 31/436

(52) U.S. Cl. 514/229.5; 514/291

(57) ABSTRACT

The present invention provides methods for treatment of neurological disorders or complications due to stroke or head injury; benign or malignant neoplastic disease, carcinomas and adenocarcinomas; proliferative disorders; and inflammatory disorders, comprising administering a compound as described herein to a subject in need thereof, and a pharmaceutically acceptable carrier, within a therapeutic window that is from about 4 hours to 24 hours, or longer, for example at least 4, 6, 9, 12, 15, 18, 21 or 24 hours, or longer, after the onset of the neurological, proliferative, or inflammatory disorder or a symptom thereof. In some embodiments, the compounds of the following structure, wherein R₁, R₂, R₄, R_{4′}, R₆, R₇, and R₁₅ are as defined herein:

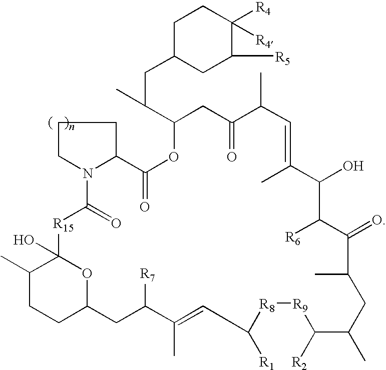

### CROSS-REFERENCE TO RELATED APPLICATIONS

This application claims the benefit of U.S. Provisional Application No. 61/029,783, filed Feb. 19, 2008, the entire disclosure of which is incorporated by reference herein.

### BACKGROUND OF THE INVENTION

The present invention provides methods for the use of rapamycin analogues in the treatment of neurological disorders that cause or involve impaired neurological function, for example impaired brain function. Nonlimiting examples of such disorders include those of idiopathic, genetic, and traumatic origin, and/or those that involve an ischemic, hemorrhagic and/or traumatic event, for example and without limitation, stroke, spinal cord injury, head trauma, traumatic brain injury, hereditary cerebral hemorrhage with amyloidosis of the Dutch type, and mild cognitive impairment caused by stroke. In some embodiments, the neurological disorders involve an ischemic or hemorrhagic event, such as stroke or trauma, which results in impaired neurological function. In some embodiments, the methods comprise administering a compound as described herein to a subject in need thereof within a therapeutic window that is from about 4 to 24 hours, or longer, for example at least 4, 6, 9, 12, 15, 18, 21 or 24 hours, or longer, after the onset of the neurological disorder or a symptom thereof.

Ischemic stroke, which accounts for 83% of all stroke cases (the remaining 17% are of the hemorrhagic-type) occurs in approximately 700,000 Americans each year, which equates to roughly 1 stroke every 45 seconds. Ischemic strokes occur as a result of an obstruction within a blood vessel supplying blood to the brain. The underlying condition for this type of obstruction is the development of fatty deposits lining the vessel walls, called atherosclerosis. These fatty deposits can cause two types of obstruction: 1) cerebral thrombosis, which refers to a thrombus (blood clot) that develops at the clogged part of the vessel and 2) cerebral embolism, which refers generally to a blood clot that forms at another location in the circulatory system, usually the heart and large arteries of the upper chest and neck. A portion of the blood clot breaks loose, enters the bloodstream and travels through the brain's blood vessels until it reaches vessels too small to let it pass. Current therapies to treat ischemic stroke are limited. To date, the only approved drug for ischemic stroke is recombinant tissue plasminogen activator (rt-PA). rt-PA, which acts as a thrombolytic, has a limited therapeutic window of opportunity (3 hours), therefore allowing only 1-2% of all stroke patients to receive treatment. Currently, there is a marketed need for neuroprotectants agents for ischemic stroke.

Given their clinical importance, prototypical molecules that clearly exhibit both neuroprotective and/or neuroregenerative activities have been highly sought after. Neurotrophins are a family of proteins that have extraordinary therapeutic properties in pre-clinical models of neurodegeneration. Although experimentally promising, clinical development of neurotrophins was met with severe obstacles and setbacks, such as the inability to deliver these large proteins to target population of neurons, instability of the proteins, and non-specific activity.

There is a great need for compounds useful in treating neurological disorders, and especially for such compounds that have a greater therapeutic window of opportunity than those treatments currently available, to afford treatment of more patients. This invention is directed to these, as well as other, important ends.

### SUMMARY OF THE INVENTION

In one aspect, the present invention provides methods of treating neurological disorders comprising administering to a subject in need thereof a compound of Formula I, and a pharmaceutically acceptable carrier, where the compound of Formula I has the structure:

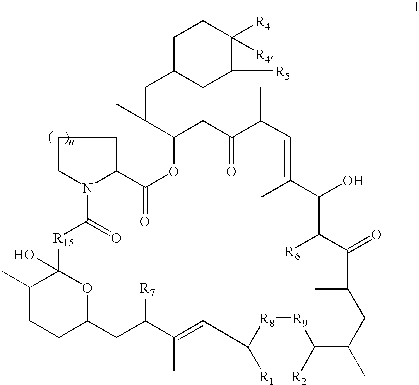

wherein the constituent variables are as defined infra, wherein said compound is administered to said subject at least 4, 6, 9, 12, 15, 18, 21 or 24 hours or longer after the onset of said disorder or a symptom thereof.

In some embodiments, the compound of Formula I has the Formula Ia:

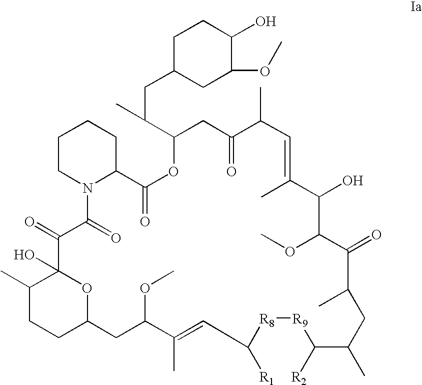

### or Ib:

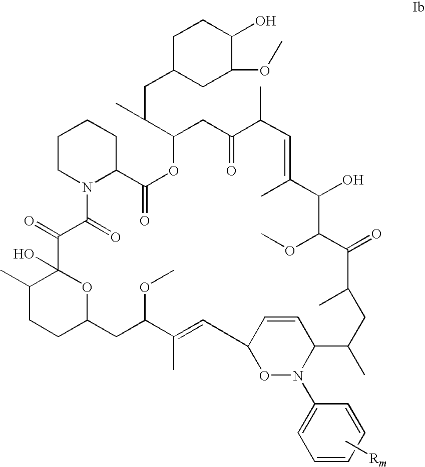

wherein the constituent variables are as defined infra.

In some embodiments, the present invention provides methods for substantially restoring brain function in a subject to where it was before the onset of a neurological disorder, comprising administering to the subject a compound of Formula I, Ia or Ib, at least 4, 6, 9, 12, 15 18, 21 or 24 hours or longer after the onset of the neurological disorder, or of a symptom thereof.

The methods of the invention are applicable to any of a variety of such disorders. Non-limiting examples of such disorders include those that cause or involve an ischemic or hemorrhagic event that results in impaired neurological function, for example impaired brain function.

Examples of disorders amenable to the methods of the invention include those of idiopathic, genetic, and traumatic origin, such as stroke, spinal cord injury, traumatic brain injury, hereditary cerebral hemorrhage with amyloidosis of the Dutch type, and mild cognitive impairment caused by stoke. Further examples include venous cardiovascular thromboembolic disorders, thromboembolic disorders in the chambers of the heart, atherosclerosis, restenosis, peripheral arterial disease, coronary bypass grafting surgery, carotid artery disease, arteritis, ischemic heart disease, cardiac ischemia, ischemia, ischemic sudden death, transient ischemic attack, stroke, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, thrombosis, supraventricular arrhythmia, atrial arrhythmia, atrial flutter, and atrial fibrillation.

Accordingly, in some embodiments, the invention provides methods for substantially restoring brain function to where it was before the onset of a neurological disorder and/or for reversing ischemia, comprising administering to a subject in need thereof a compound of Formula I, Ia or Ib, at least 4, 6, 9, 12, 15 18, 21 or 24 hours or longer after the onset of impairment of brain function or the ischemia, or a symptom thereof.

In some further embodiments, the invention provides methods of treating complications due to stroke, head trauma, spinal cord injury, or traumatic brain injury, comprising administering to a subject in need thereof a compound of Formula I, Ia or Ib, at least 4, 6, 9, 12, 15 18, 21 or 24 hours or longer after the onset of the stroke, head trauma, spinal cord injury, or traumatic brain injury.

In some further embodiments, the invention provides methods for treating a disorder selected from venous cardiovascular thromboembolic disorders, thromboembolic disorders in the chambers of the heart, atherosclerosis, restenosis, peripheral arterial disease, coronary bypass grafting surgery, carotid artery disease, arteritis, ischemic heart disease, cardiac ischemia, ischemia, ischemic sudden death, transient ischemic attack, stroke, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, thrombosis, supraventricular arrhythmia, atrial arrhythmia, atrial flutter, and atrial fibrillation, comprising administering to a subject in need thereof a compound of Formula I, Ia, or Ib, at least 4, 6, 9, 12, 15, 18, 21 or 24 hours, or longer, after the onset of said disorder or a symptom thereof.

In some embodiments of the methods of the invention, the compound of Formula I is prepared using norrapamycin, deoxorapamycin, or desmethylrapamycin as starting materials (see FIG. 1).

In some embodiments the starting material is 9-deoxorapamycin.

In other embodiments the starting material is selected from 32-O-desmethylrapamycin, 16-O-desmethylrapamycin, 27-O-desmethylrapamycin, and 39-O-desmethylrapamycin.

Other aspects and advantages of the present invention are described further in the following detailed description of the embodiments thereof.

### BRIEF DESCRIPTION OF THE FIGURES

FIG. 1 shows examples of rapamycin and rapamycin analogues that can be used as starting materials to produce the compounds of Formula I.

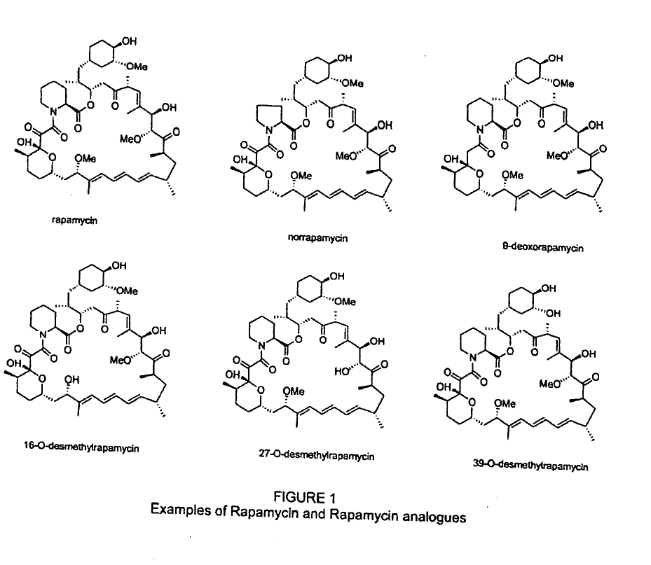

FIG. 2 provides the nuclear magnetic resonance (NMR) spectra for the compound of Example 1. The NMR spectra were obtained in d₃-acetonitrile using a 400 MHz spectrometer.

FIG. 3 provides the NMR spectra for the compound of Example 2. The NMR spectra were obtained in d₃-acetonitrile using a 400 MHz spectrometer.

FIG. 4 provides the NMR spectra for the compound of Example 3. The NMR spectra were obtained in d₃-acetonitrile using a 400 MHz spectrometer.

FIG. 5 provides the NMR spectra for the compound of Example 4. The NMR spectra were obtained in d₃-acetonitrile using a 400 MHz spectrometer.

FIG. 6 provides the NMR spectra for the compound of Example 5. The NMR spectra were obtained in d₃-acetonitrile using a 400 MHz spectrometer.

FIG. 7 provides the NMR spectra for the compound of Example 6. The NMR spectra were obtained in d₃-acetonitrile using a 400 MHz spectrometer.

FIG. 8 provides data showing that the Compound of Example 2 improves neurological deficits following tMCAO in rats.

FIG. 9 provides data showing that the Compound of Example 2 attenuates weight loss following tMCAO in rats.

FIG. 10 provides data showing that the Compound of Example 2 reduces infarct volume following tMCAO in rats.

FIG. 11 provides data showing that the Compound of Example 2 (2 hour post-occlusion) improves neurological function following pMCAO (3-weeks) in rats.

FIG. 12 provides data showing that the Compound of Example 2 (6 hour post-occlusion) improves neurological function following pMCAO (3-weeks) in rats.

FIG. 13 provides data showing that the Compound of Example 2 (6 hour post-occlusion) improves neurological function following pMCAO (3 months) in rats.

FIG. 14 provides data showing that the Compound of Example 2 (24 hour post-occlusion) improves neurological function following pMCAO (3 weeks) in rats.

FIG. 15 provides data showing that the Compound of Example 2 promotes neuroregeneration in the hippocampus.

FIG. 16 provides data showing that the Compound of Example 2 promotes neuroregeneration in the cortex.

### DETAILED DESCRIPTION OF THE INVENTION

The present invention provides methods for treating neurological disorders, and symptoms of neurological disorders, including stroke, head trauma, and complications due to the preceding, that include administering a compound of the invention to a subject in need of such treatment within a therapeutic window of opportunity that is from about 4 hours to about 24 hours or longer. The compound of the invention can be administered at least 4, 6, 9, 12, 15, 18, 21 or 24 hours, or longer, after the onset of the condition or disorder to be treated, or the onset of a symptom thereof.

The rapamycin analogues of Formula I are useful as neuroprotective, anti-proliferative, and/or anti-inflammatory agents, as described in U.S. Pat. No. 7,273,874, which is incorporated by reference herein in its entirety. The compounds of formula I are useful as neuroprotective agents in compositions for use in treating neurological disorders, including, e.g., a neurodegenerative or neuromuscular degenerative condition, that can be a result of a genetic disorder present at birth, a disorder developed during the lifespan of an individual such as stroke, and/or the result of physical trauma, e.g., head injury, spinal injury, or injury to the peripheral nervous system.

The compound of Example 2 herein, a representative compound of formula I, possesses a therapeutic window of opportunity for treatment of stroke of at least 24 hours; i.e., it can exert its therapeutic effect with administration commencing at a significant time after the onset of stroke, for example at least 4, 6, 9, 12, 15, 18, 21 or 24 hours or longer after the onset of stroke. Thus, a compound of the invention may be useful in ameliorating the symptoms of a pre-existing neurological disorder, preventing further neuro- and/or neuromuscular degeneration, by initial administration at a time following onset of the neurological disorder of up to at least 24 hours.

### I. Compounds of Formula I

The methods of the present invention relate to rapamycin analogues of the formula I:

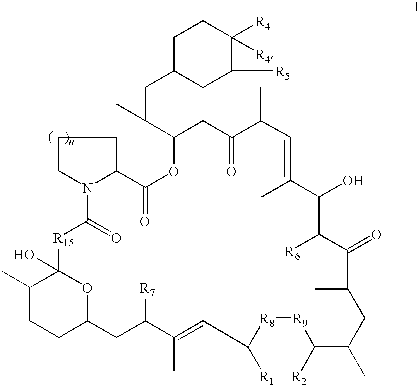

R₁ and R₂ in the above-noted formula are different, independent groups and are selected from among OR₃ and N(R_{3′})(R_{3″}) or R₁ and R₂ are different, are connected through a single bond, and are selected from O and NR₃. R₃, R_{3′}, and R_{3″} are independently selected from among H, C₁₋₆alkyl, substituted C₁₋₆alkyl, C₃₋₈cycloalkyl, substituted C₃₋₈cycloalkyl, C₆₋₂₀aryl, substituted aryl, heteroaryl, and substituted heteroaryl. R₄ and R_{4′} are (a) independently selected from among H, OH, O(C₁₋₆alkyl), O(substituted C₁₋₆alkyl), O(acyl), O(C₆₋₂₀aryl), O(substituted aryl), and halogen; or (b) taken together to form a double bond to 0 (i.e. ═O). R₅, R₆, and R₇ are independently selected from among H, OH, and OCH₃. R₈ and R₉ are connected through a (i) single bond and are CH₂ or (ii) double bond and are CH. R₁₅ is selected from among C(═O), CHOH, and CH₂ and n is 1 or 2; or pharmaceutically acceptable, salts, prodrugs, or metabolites thereof.

In further embodiments, R₁ and R₂ are connected through a single bond and are selected from O and NR₃. In still a further embodiment, R₁ is O and R₂ is NR₃.

In one embodiment, R_{3′} or R_{3″} is a C₆₋₂₀aryl or substituted aryl group, or a substituted benzene ring. In another embodiment, substituted benzene groups at R_{3′} or R_{3″} include rings of the following structure:

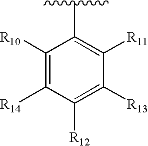

R₁₀, R₁₁, R₁₂, R₁₃, and R₁₄ are independently selected from among H, C₁₋₆ alkyl, substituted C₁₋₆alkyl, C₆₋₂₀aryl, substituted aryl, heteroaryl, substituted heteroaryl, halogen, acyl, OH, O(C₁₋₆alkyl), O(substituted alkyl), O(C₆₋₂₀aryl), O(substituted aryl), O(acyl), NH₂, NH(C₁₋₆alkyl), NH(substituted alkyl), NH(C₆₋₂₀aryl), NH(substituted aryl), and NH(acyl).

In further embodiments, R₃, R_{3′} or R_{3″} are phenyl optionally substituted by 1 or 2 substituents selected from C₁₋₆alkyl and halogen. In still further embodiments, R₃, R_{3′} or R_{3″} are phenyl optionally substituted with 1 or 2 methyl or chloro substituents, e.g. phenyl and 3-methyl, 4-chlorophenyl.

In one embodiment, R₄ or R_{4′} are OH or O(acyl), e.g., where the acyl is —C(O)— optionally substituted alkyl, in particular where alkyl can be straight or branched and optionally substituted e.g. by heterocyclic such as aromatic heterocyclic such as pyridyl. An example is:

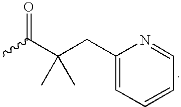

In other embodiments, rapamycin analogues of formula I include those where R₅, R₆ and R₇ are OCH₃, those where the nitrogen containing ring at positions 17-22 of the rapamycin backbone is a piperidine ring, or where R₁₅ is a carbonyl.

In one embodiment, the compounds of formula I have the following formula Ia:

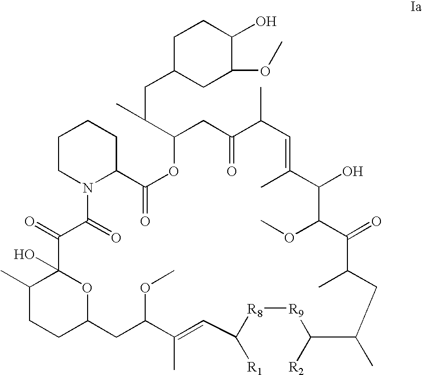

where R₁, R₂, R₈, and R₉ are defined as noted above.

In another embodiment, the compounds of formula I have the following formula Ib:

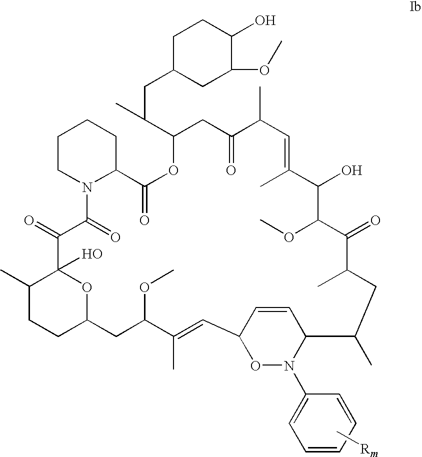

In formula Ib, R is independently selected from among H, C₁₋₆alkyl, substituted C₁₋₆alkyl, C₆₋₂₀aryl, substituted aryl, heteroaryl, substituted heteroaryl, halogen, acyl, OH, O(C₁₋₆alkyl), O(substituted alkyl), O(C₆₋₂₀aryl), O(substituted aryl), O(acyl), NH₂, NH(C₁₋₆alkyl), NH(substituted alkyl), NH(C₆₋₂₀aryl), NH(substituted aryl), and NH(acyl) and m is 1 to 5.

Examples of compounds of formula I are illustrated herein and include:

### Example 1

9,27-dihydroxy-3-{2-[4-hydroxy-3-methoxycyclohexyl]-1-methylethyl}-10,21-dimethoxy-6,8,12,14,20,26-hexamethyl-37-phenyl-4,9,10,12,13,14,15,18,21,22,23,24,25,26,27,32,33,34,34a-nonadecahydro-3H-23,27-epoxy-18,15-(epoxyimino)pyrido[2,1-c][1,4]oxazacyclohentriacontine-1,5,11,28,29(6H,31H)-pentone;

### Example 2

9,27-dihydroxy-3-{2-[4-hydroxy-3-methoxycyclohexyl]-1-methylethyl}-10,21-dimethoxy-6,8,12,14,20,26-hexamethyl-37-phenyl-4,9,10,12,13,14,15,16,17,18,21,22,23,24,25,26,27,32,33,34,34a-henicosahydro-3H-23,27-epoxy-18,15-(epoxyimino)pyrido[2,1-c][1,4]oxazacyclohentriacontine-1,5,11,28,29(6H,31H)-pentone;

### Example 3

37-(4-chloro-3-methylphenyl)-9,27-dihydroxy-3-{-2-[4-hydroxy-3-methoxycyclohexyl]-1-methylethyl}-10,21-dimethoxy-6,8,12,14,20,26-hexamethyl-4,9,10,12,13,14,15,18,21,22,23,24,25,26,27,32,33,34,34a-nonadecahydro-3H-23,27-epoxy-18,15-(epoxyimino)pyrido[2,1-c][1,4]oxazacyclohentriacontine-1,5,11,28,29(6H,31H)-pentone;

### Example 4

37-(2,6-dichlorophenyl)-9,27-dihydroxy-3-{2-[4-hydroxy-3-methoxycyclohexyl]-1-methylethyl}-10,21-dimethoxy-6,8,12,14,20,26-hexamethyl-4,9,10,12,13,14,15,18,21,22,23,24,25,26,27,32,33,34,34a-nonadecahydro-3H-23,27-epoxy-18,15-(epoxyimino)pyrido[2,1-c][1,4]oxazacyclohentriacontine-1,5,11,28,29(6H,31H)-pentone;

### Example 5

37-(2,6-dichlorophenyl)-9,27-dihydroxy-3-{-2-[4-hydroxy-3-methoxycyclohexyl]-1-methylethyl}-10,21-dimethoxy-6,8,12,14,20,26-hexamethyl-4,9,10,12,13,14,15,18,21,22,23,24,25,26,27,32,33,34,34a-henicosahydro-3H-23,27-epoxy-18,15-(epoxyimino)pyrido[2,1-c][1,4]oxazacyclohentriacontine-1,5,11,28,29(6H,31H)-pentone;

### Example 6

9,27-dihydroxy-3-{-2-[4-hydroxy-3-methoxycyclohexyl]-1-methylethyl}-10,21-dimethoxy-6,8,12,14,20,26-hexamethyl-37-phenyl-4,9,10,12,13,14,15,18,21,22,23,24,25,26,27,32,33,34,34a-nonadecahydro-3H-23,27-epoxy-18,15-(epoxyimino) pyrido[2,1-c][1,4]oxazacyclohentriacontine-1,5,11,28,29(6H,31H)-pentone ester with -2,2-dimethyl-3′-(pyridin-2-yl)-propionic acid;

or pharmaceutically acceptable, salts, prodrugs, or metabolites thereof. The invention is not limited to these illustrative compounds.

In another embodiment, the compounds of the methods can include the following:

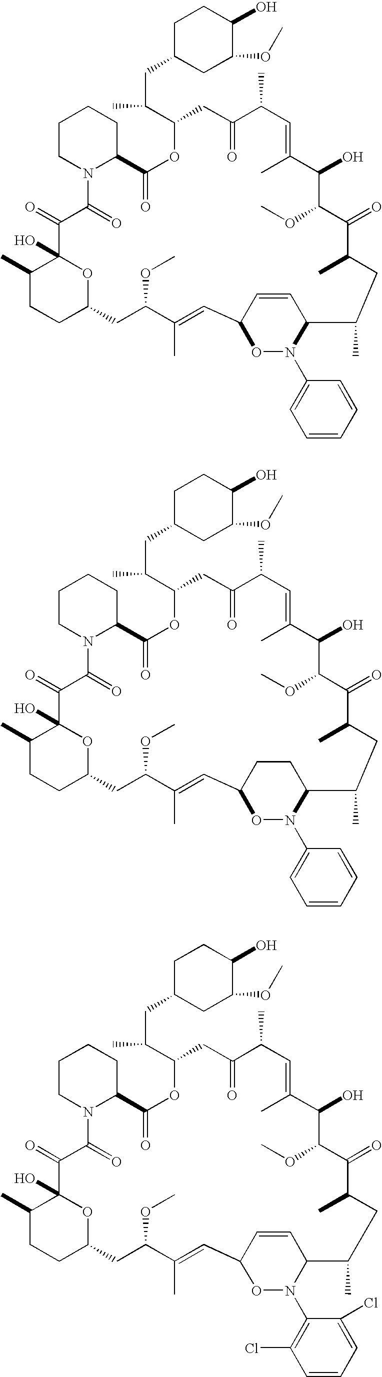

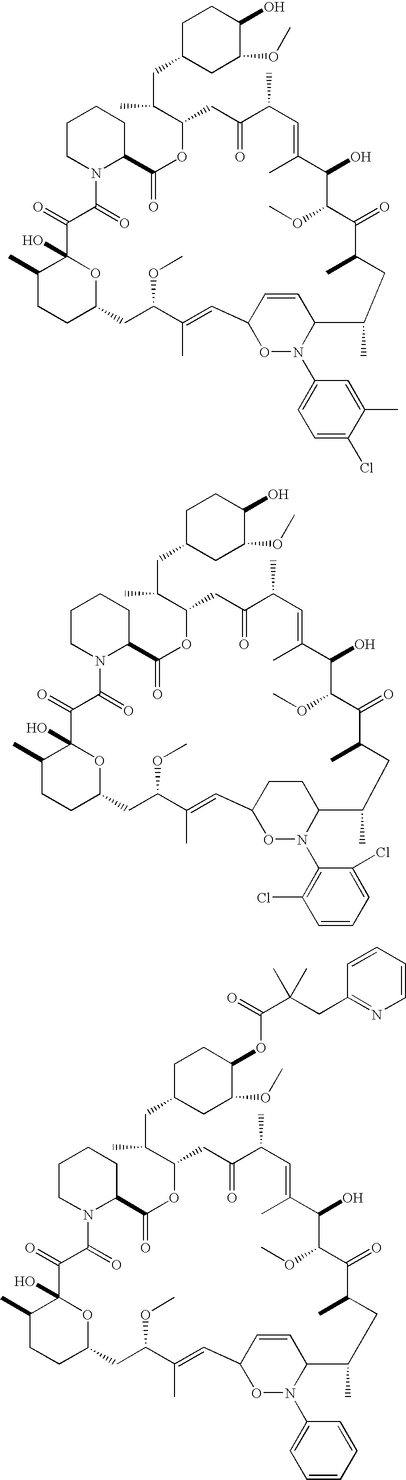

The present methods also are amenable to compounds of formula I where R₁ and R₂ are connected through a single bond; R₁ is O; R₂ is NR₃; R₃ is phenyl; R₄ is OH; R₅, R₆ and R₇ are OCH₃; and R₈ and R₉ are HC═CH; a compound where R₁ is OR₃; R₂ is N(R_{3′})(R_{3″}); R₃ is H; R_{3′} is H; R_{3″} is phenyl; R₄ is OH; R₅, R₆ and R₇ are OCH₃; and R₈ and R₉ are H₂C—CH₂; a compound where R₁ and R₂ are connected through a single bond; R₁ is O; R₂ is NR₃; R₃ is phenyl; R₄ is OH; R₅, R₆ and R₇ are OCH₃; and R₈ and R₉ are H₂C—CH₂; a compound where R₁ and R₂ are connected through a single bond; R₁ is O; R₂ is NR₃; R₄ is OH; R₅, R₆ and R₇ are OCH₃; R₈ and R₉ are HC═CH; and R₃ is

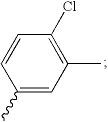

a compound where R₁ and R₂ are connected through a single bond; R₁ is O; R₂ is NR₃; R₄ is OH; R₅, R₆ and R₇ are OCH₃; R₈ and R₉ are HC═CH; and R₃ is

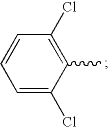

a compound where R₁ and R₂ are connected through a single bond; R₁ is O; R₂ is NR₃; R₃ is phenyl; R₅, R₆ and R₇ are OCH₃; R₈ and R₉ are HC═CH; and R₄ is

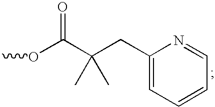

and a compound where R₁ and R₂ are connected through a single bond; R₁ is O; R₂ is NR₃; R₄ is OH; R₅, R₆ and R₇ are OCH₃; R₈ and R₉ are H₂C—CH₂; and R₃ is

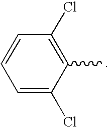

The compounds of formula I can contain one or more asymmetric carbon atoms and some of the compounds can contain one or more asymmetric (chiral) centers and can thus give rise to optical isomers and diastereomers. While shown without respect to stereochemistry, when the compounds can contain one or more chiral centers, at least one of the chiral centers may be of S-stereochemistry. Thus, the compounds of formula I include such optical isomers and diastereomers; as well as the racemic and resolved, enantiomerically pure stereoisomers; as well as other mixtures of the R and S stereoisomers, and pharmaceutically acceptable salts, hydrates, metabolites, and prodrugs thereof.

The term “alkyl” is used herein to refer to both straight- and branched-chain saturated aliphatic hydrocarbon groups having 1 to 10 carbon atoms, and can have about 1 to 8 carbon atoms. The term “alkenyl” is used herein to refer to both straight- and branched-chain alkyl groups having one or more carbon-carbon double bonds and containing 2 to about 10 carbon atoms. In one embodiment, the term alkenyl refers to an alkyl group having 1 or 2 carbon-carbon double bonds and having 2 to about 6 carbon atoms. The term “alkynyl” group is used herein to refer to both straight- and branched-chain alkyl groups having one or more carbon-carbon triple bond and having 2 to about 8 carbon atoms. In another embodiment, the term alkynyl refers to an alkyl group having 1 or 2 carbon-carbon triple bonds and having 2 to about 6 carbon atoms.

The term “cycloalkyl” is used herein to refer to an alkyl group as previously described that is cyclic in structure and has 3 to about 10 carbon atoms, or about 5 to about 8 carbon atoms.

The terms “substituted alkyl”, “substituted alkenyl”, and “substituted alkynyl” refer to alkyl, alkenyl, and alkynyl groups, respectively, having one or more substituents including, without limitation, halogen, CN, OH, NO₂, amino, C₆₋₂₀aryl, heterocyclic, C₁₋₆alkoxy, aryloxy, alkylcarbonyl, alkylcarboxy, and arylthio, which groups can be optionally substituted e.g. by 1 to 4 substituents including halogen, CN, OH, NO₂, amino, C₁₋₆alkyl, C₄₋₁₀cycloalkyl, C₂₋₁₀alkenyl, C₂₋₈alkynyl, C₁₋₆alkoxy, aryloxy, alkyloxy, alkylcarbonyl, alkylcarboxy, aminoalkyl, and arylthio. These substituents can be attached to any carbon of an alkyl, alkenyl, or alkynyl group provided that the attachment constitutes a stable chemical moiety.

The term “aryl” as used herein refers to an aromatic system, e.g., of 6-20 carbon atoms, which can include a single ring or multiple aromatic rings fused or linked together (e.g. two or three) where at least one part of the fused or linked rings forms the conjugated aromatic system. The aryl groups can include, but are not limited to, phenyl, naphthyl, biphenyl, anthryl, tetrahydronaphthyl, phenanthryl, indene, benzonaphthyl, fluorenyl, and carbazolyl.

The term “substituted aryl” refers to an aryl group which is substituted with one or more substituents including halogen, CN, OH, NO₂, amino, alkyl, C₄₋₁₀cycloalkyl, C₂₋₁₀alkenyl, C₂₋₈alkynyl, C₁₋₆alkoxy, aryloxy, alkyloxy, alkylcarbonyl, alkylcarboxy, aminoalkyl, and arylthio, which groups can be optionally substituted. In one embodiment, a substituted aryl group is substituted with 1 to 4 substituents including halogen, CN, OH, NO₂, amino, alkyl, C₄₋₁₀cycloalkyl, C₂₋₁₀alkenyl, C₂₋₈alkynyl, C₁₋₆alkoxy, aryloxy, alkyloxy, alkylcarbonyl, alkylcarboxy, aminoalkyl, and arylthio.

The term “heterocyclic” as used herein refers to a stable 4- to 7-membered monocyclic or multicyclic heterocyclic ring, which is saturated, partially unsaturated, or wholly unsaturated, including aromatic such as pyridyl. The heterocyclic ring has carbon atoms and one or more heteroatoms including nitrogen, oxygen, and sulfur atoms. In one embodiment, the heterocyclic ring has 1 to 4 heteroatoms in the backbone of the ring. When the heterocyclic ring contains nitrogen or sulfur atoms in the backbone of the ring, the nitrogen or sulfur atoms can be oxidized. The term “heterocyclic” also refers to multicyclic rings, e.g., of 9 to 20 ring members in which a heterocyclic ring is fused to an aryl ring. The heterocyclic ring can be attached to the aryl ring through a heteroatom or carbon atom, provided the resultant heterocyclic ring structure is chemically stable.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Oxygen-containing rings include, but are not limited to, furyl, tetrahydrofuranyl, pyranyl, pyronyl, and dioxinyl rings. Nitrogen-containing rings include, without limitation, pyrrolyl, pyrazolyl, imidazolyl, triazolyl, pyridyl, piperidinyl, 2-oxopiperidinyl, pyridazinyl, pyrimidinyl, pyrazinyl, piperazinyl, azepinyl, triazinyl, pyrrolidinyl, and azepinyl rings. Sulfur-containing rings include, without limitation, thienyl and dithiolyl rings. Mixed heteroatom containing rings include, but are not limited to, oxathiolyl, oxazolyl, thiazolyl, oxadiazolyl, oxatriazolyl, dioxazolyl, oxathiazolyl, oxathiolyl, oxazinyl, oxathiazinyl, morpholinyl, thiamorpholinyl, thiamorpholinyl sulfoxide, oxepinyl, thiepinyl, and diazepinyl rings. Fused heteroatom-containing rings include, but are not limited to, benzofuranyl, thionapthene, indolyl, benazazolyl, purindinyl, pyranopyrrolyl, isoindazolyl, indoxazinyl, benzoxazolyl, anthranilyl, benzopyranyl, quinolinyl, isoquinolinyl, benzodiazonyl, naphthylridinyl, benzothienyl, pyridopyridinyl, benzoxazinyl, xanthenyl, acridinyl, and purinyl rings.

The term “substituted heterocyclic” as used herein refers to a heterocyclic group having one or more substituents including halogen, CN, OH, NO₂, amino, C₁₋₆ alkyl, C₄₋₁₀cycloalkyl, C₂₋₁₀alkenyl, C₂₋₈alkynyl, C₁₋₆alkoxy, aryloxy, alkyloxy, alkylcarbonyl, alkylcarboxy, aminoalkyl, and arylthio, which groups can be optionally substituted. In one embodiment, a substituted heterocyclic group is substituted with 1 to 4 substituents.

The term “acyl” refers to a —C(O)— group, which is substituted at the carbon atom. The acyl group can be substituted or a terminal acyl group such as an HC(O)— group. The substituents can include any substituents noted above for alkyl groups, viz. one or more substituents including, without limitation, halogen, CN, OH, NO₂, amino, C₆₋₂₀aryl, heterocyclic, C₁₋₆alkoxy, aryloxy, alkylcarbonyl, alkylcarboxy, and arylthio, which groups can be optionally substituted. Examples include —C(O)— alkoxy (e.g. —OMe or —OEt) or —C(O)-alkyl where alkyl can be straight or branched and optionally substituted e.g., by heterocyclic (such as pyridyl).

The term “alkoxy” as used herein refers to the O(C₁₋₆alkyl) group, where the point of attachment is through the oxygen-atom and the alkyl group is optionally substituted.

The term “aryloxy” as used herein refers to the O(C₆₋₂₀aryl) group, where the point of attachment is through the oxygen-atom and the aryl group is optionally substituted.

The term “alkyloxy” as used herein refers to the C₁₋₆alkylOH group, where the point of attachment is through the alkyl group.

The term “arylthio” as used herein refers to the S(C₆₋₂₀aryl) group, where the point of attachment is through the sulfur-atom and the aryl group can be optionally substituted.

The term “alkylcarbonyl” as used herein refers to the C(═O)(C₁₋₆alkyl) group, where the point of attachment is through the carbon-atom of the carbonyl moiety and the alkyl group is optionally substituted.

The term “alkylcarboxy” as used herein refers to the C(═O)O(C₁₋₆alkyl) group, where the point of attachment is through the carbon-atom of the carboxy moiety and the alkyl group is optionally substituted.

The term “aminoalkyl” as used herein refers to both secondary and tertiary amines where the point of attachment is through the nitrogen-atom and the alkyl groups are optionally substituted. The alkyl groups can be the same or different.

The term “halogen” as used herein refers to Cl, Br, F, or I groups.

### II. Methods of Preparing the Compounds of Formula I

The rapamycin analogues of formula I of the methods of the present invention are prepared from a rapamycin starting material. The rapamycin starting material includes, without limitation, rapamycin, norrapamycin, deoxorapamycin, desmethylrapamycins, and desmethoxyrapamycin, or pharmaceutically acceptable salts, prodrugs, or metabolites thereof. One of skill in the art would readily be able to select a suitable rapamycin starting material that can be utilized to prepare the rapamycin analogues of the present invention.

The term “deoxorapamycin” refers to a class of rapamycin compounds including, but not limited to 9-deoxorapamycin.

The term “desmethylrapamycin” refers to the class of rapamycin compounds that lack one or more methyl groups. Examples of desmethylrapamycins include 3-desmethylrapamycin (U.S. Pat. No. 6,358,969), 7-O-desmethyl-rapamycin (U.S. Pat. No. 6,399,626), 17-desmethylrapamycin (U.S. Pat. No. 6,670,168), 32-O-desmethylrapamycin, 16-O-desmethylrapamycin, 27-O-desmethylrapamycin, and 39-O-desmethylrapamycin, among others.

The term “desmethoxyrapamycin” refers to the class of rapamycin compounds, which lack one or more methoxy groups, and includes, without limitation, 32-desmethoxyrapamycin.

The rapamycin analogues of formula I can be prepared by combining a rapamycin starting material and a dienophile. The term “dienophile” refers to a molecule that reacts with a 1,3-diene to give a [4+2]cycloaddition product. The dienophile can be an optionally substituted nitrosobenzene. A variety of nitrosobenzenes can be used as the dienophile, such as, 2,6-dichloronitrosobenzene, and 1-chloro-2-methyl-4-nitrosobenzene, among others. One of skill in the art would readily be able to select the amount of nitrosobenzene that would be effective in preparing the rapamycin analogues. An excess of the nitrosobenzene can be used, such as in a 5:1 ratio of nitrosobenzene to rapamycin starting material and a 1:1, 2:1, or 3:1 ratio of nitrosobenzene to rapamycin can be used.

The nitrosobenzene and rapamycin starting material is combined in a solvent. The solvent can dissolve the nitrosobenzene and/or rapamycin on contact, or dissolve the nitrosobenzene and rapamycin as the reaction proceeds. Solvents that can be utilized in the present invention include, without limitation, dimethylformamide, dioxane such as p-dioxane, chloroform, alcohols such as methanol and ethanol, ethyl acetate, water, acetonitrile, tetrahydrofuran, dichloromethane, and toluene, or combinations thereof. One of skill in the art would readily be able to select a suitable solvent based upon the solubility of the rapamycin starting material and nitrosobenzene, as well as the reactivity of the solvent with the same. The amount of solvent utilized depends upon the scale of the reaction and the amount of rapamycin starting material and nitrosobenzene present in the reaction mixture. One of skill in the art would readily be able to determine the amount of solvent required.

The solution containing the nitrosobenzene, rapamycin starting material and solvent is maintained at elevated temperatures, and preferably a temperature that does not promote decomposition of the rapamycin and nitrosobenzene. In one embodiment, the solution is maintained at a temperature of about 30° C. to about 70° C., and can be about 50° C. The components are heated for a period of time sufficient to permit reaction between the rapamycin and nitrosobenzene. One of skill in the art using known techniques would readily be able to monitor the progress of the reaction during heating and thereby determine the amount of time required to perform the reaction. In one embodiment, the rapamycin and nitrosobenzene are combined with p-dioxane and maintained at a temperature of about 50° C.

Isolation and purification of the rapamycin analogue can be accomplished using chromatography including, without limitation, recrystallization, high performance liquid chromatography (HPLC), such as, reverse phase HPLC and normal phase HPLC, and size-exclusion chromatography.

Once the rapamycin analogue is obtained, it can be reduced to form a more saturated rapamycin analogue and reduction of the rapamycin analogue can be effected using a hydrogenation agent. One of skill in the art would readily be able to select a suitable hydrogenation agent for use in the present invention. Typically, transition metal catalysts or transition metals on a support, such as a carbon support among others, in the presence of hydrogen gas, are utilized to carry out the reduction. In one embodiment, the reduction is performed using palladium metal on carbon in the presence of hydrogen gas.

Reduction of the rapamycin analogue is typically carried out in a solvent. A variety of solvents can be utilized in the reduction and include, without limitation, alcohols such as methanol. One of skill in the art would readily be able to select a suitable solvent for use in the present invention and depending on the hydrogenation catalyst and rapamycin analogue being reduced. The amount of solvent depends on the scale of the reaction, and the amount of rapamycin analogue being reduced.

One of skill in the art would be able to determine and adjust the amount of hydrogenation agent necessary to perform the reduction and to form the more saturated rapamycin analogues. Further, a variety of apparatuses can be utilized to perform the hydrogenation and include Parr apparatuses, among others. The selection of the particular apparatus for the hydrogenation is well within one of skill in the art.

One method of preparing the rapamycin analogues of formula I is summarized in Scheme 1 below:

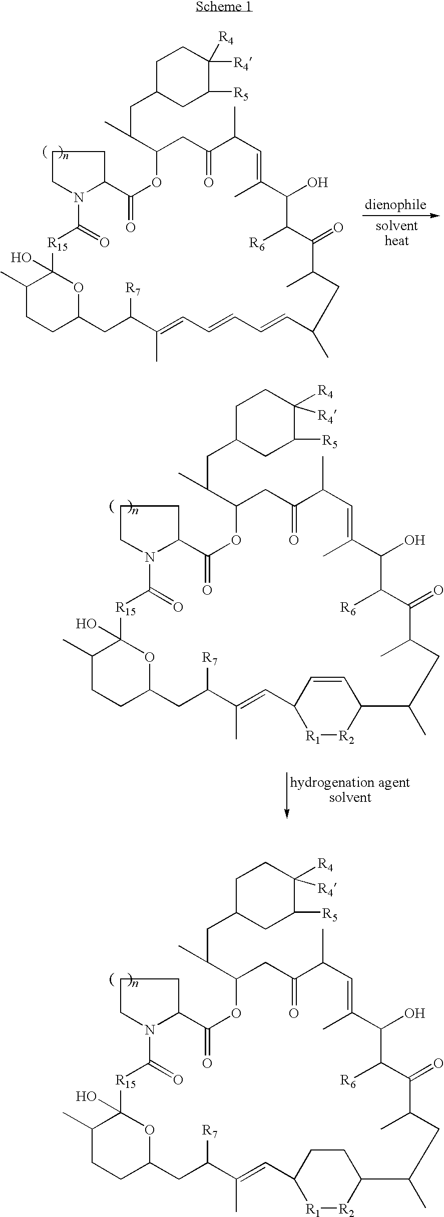

where R₁, R₂, R₄, R_{4′}, R₆, R₇, R₁₅, and n are defined above.

The rapamycin analogues of formula I can be utilized in the form of pharmaceutically acceptable salts, prodrugs, or metabolites thereof derived from pharmaceutically or physiologically acceptable acids or bases. These salts include, but are not limited to, the following salts with mineral or inorganic acids such as hydrochloric acid, sulfuric acid, nitric acid, phosphoric acid and organic acids such as acetic acid, oxalic acid, succinic acid, and maleic acid. Other salts include salts with alkali metals or alkaline earth metals, such as sodium, potassium, calcium or magnesium in the form of esters, carbamates and other conventional “pro-drug” forms, which, when administered in such form, convert to the active moiety in vivo.

### III. Methods of Using the Compounds of Formula I

The rapamycin analogues of formula I, Ia, and Ib, are useful in the treatment of neurological disorders (including neuromuscular disorders) and the other disorders described herein. In some embodiments, the rapamycin analogues of formula I, Ia, and Ib, are useful when administered within a therapeutic window that is from 4 to 24 hours, or longer, for example 4, 6, 9, 12, 15, 18, 21 or 24 hours, or longer, after the onset of the disorder as described herein.

The rapamycin analogues of formula I, Ia, and Ib, including the more and less saturated rapamycin analogues, are useful in disorders involving the dysfunction of calcium (Ca²⁺) ion channels, such as ryanodine receptor (RyR1, RyR2, and Ryr3) channelopathies including, among others, malignant hyperthermia, central core disease, cathecolaminergic polymorphic ventricular tachycardia, and arrhythmogenic right ventricular dysplasia type 2 (ARVD-2). The rapamycin analogues of formula I, Ia, and Ib of the present invention, including the more and less saturated rapamycin analogues, are also useful in dihydropyridine receptor channelopathies, including those resultant from ryanodine receptor activity due to the activity of dihydropyridine-sensitive calcium ion (Ca²⁺) channels. As used herein, the term “channelopathy” refers to a disease or disorder involving dysfunction of an ion channel.

The diseases and disorders referred to herein are grouped herein under conventional headings, e.g., neurological disorders. One of skill in the art will recognize that the diseases or disorders referred to herein may be appropriately grouped under different headings or under multiple headings. The grouping of diseases and/or disorders referred to herein is not a limitation of the present invention.

The compounds of formula I are useful in treating neurological disorders, including those that cause or involve impaired neurological function, for example impaired brain function. Nonlimiting examples of such disorders include those of idiopathic, genetic, and traumatic origin, and/or those that involve an ischemic, hemorrhagic and/or traumatic event, for example and without limitation, stroke, spinal cord injury, head trauma, traumatic brain injury, hereditary cerebral hemorrhage with amyloidosis of the Dutch type, and mild cognitive impairment caused by stoke. Further examples of neurological disorders amenable to the methods disclosed herein include venous cardiovascular thromboembolic disorders, thromboembolic disorders in the chambers of the heart, atherosclerosis, restenosis, peripheral arterial disease, coronary bypass grafting surgery, carotid artery disease, arteritis, ischemic heart disease, cardiac ischemia, ischemia, ischemic sudden death, transient ischemic attack, stroke, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, thrombosis, supraventricular arrhythmia, atrial arrhythmia, atrial flutter, and atrial fibrillation.

The present methods include the use of the compounds of formula I, within the disclosed therapeutic window, for treating complications due to the above disorders, and other injuries to the brain, peripheral nervous, central nervous, or neuromuscular system, and in the preparation of medicaments therefore.

The rapamycin analogues are also useful as neuroprotective agents, i.e., restoring some neurological and/or neuromuscular or other function following onset of one of the above conditions and/or injury, stroke, or other trauma.

The present compounds are useful in treating cardiovascular disorders including, but not limited to: thromboembolic disorders, including arterial cardiovascular thromboembolic disorders, venous cardiovascular thromboembolic disorders, and thromboembolic disorders in the chambers of the heart; atherosclerosis; restenosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); first or recurrent myocardial infarction; acute myocardial infarction (AMI); myocardial infarction; non-Q wave myocardial infarction; non-STE myocardial infarction; coronary artery disease; ischemic heart disease; cardiac ischemia; ischemia; ischemic sudden death; transient ischemic attack; stroke; peripheral occlusive arterial disease; venous thrombosis; deep vein thrombosis; thrombophlebitis; arterial embolism; coronary arterial thrombosis; cerebral arterial thrombosis; cerebral embolism; kidney embolism; pulmonary embolism; thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis; thrombosis resulting from atherosclerosis, surgery or surgical complications, prolonged immobilization, arterial fibrillation, congenital thrombophilia, cancer, diabetes, effects of medications or hormones, and complications of pregnancy; cardiac arrhythmias including supraventricular arrhythmias, atrial arrhythmias, atrial flutter, atrial fibrillation; other diseases listed in Heart Disease: A Textbook of Cardiovascular Medicine, 2 Volume Set, 6th Edition, 2001, Eugene Braunwald, Douglas P. Zipes, Peter Libby, Douglas D. Zipes; and in the preparation of medicaments therefore.

In a further embodiment, the cardiovascular disease is: atherosclerosis; coronary heart disease (CHD); restenosis; peripheral arterial disease; coronary bypass grafting surgery; carotid artery disease; arteritis; myocarditis; cardiovascular inflammation; vascular inflammation; unstable angina (UA); unstable refractory angina; stable angina (SA); chronic stable angina; acute coronary syndrome (ACS); myocardial infarction; or acute myocardial infarction (AMI), including first or recurrent myocardial infarction, non-Q wave myocardial infarction, non-ST-segment elevation myocardial infarction and ST-segment elevation myocardial infarction.

In still a further embodiment, the cardiovascular disease is: atherosclerosis; coronary heart disease (CHD); unstable angina (UA); unstable refractory angina; stable angina (SA); chronic stable angina; acute coronary syndrome (ACS); myocardial infarction; or acute myocardial infarction (AMI), including first or recurrent myocardial infarction, non-Q wave myocardial infarction, non-ST-segment elevation myocardial infarction and ST-segment elevation myocardial infarction.

The dosage requirements of the rapamycin analogues of the present invention can vary depending on the condition, severity of the symptoms presented and the particular subject being treated. One of skill in the art would readily be able to determine the amount of the rapamycin analogue required. In one embodiment, about 0.5 milligram (mg) to about 200 mg is administered. In a further embodiment, about 0.5 mg to about 100 mg is administered. In another embodiment, about 0.5 mg to about 75 mg is administered. In yet a further embodiment, about 1 mg to about 25 mg is administered. In another embodiment, about 0.5 mg to about 10 mg is administered, particularly when used in combination with another agent. In yet a further embodiment, about 2 mg to about 5 mg is administered. In yet another embodiment, about 5 mg to about 15 mg is administered.

Treatment can be initiated with dosages of the rapamycin analogue smaller than those required to produce a desired effect and may be less than the optimum dose of the rapamycin analogue. Thereafter, the dosage can be increased until the optimum effect under the circumstances is reached. Precise dosages will be determined by the administering physician based on experience with the individual subject being treated. In general, the compositions are administered at a concentration that will afford effective results without causing any harmful or deleterious side effects.

### IV. Methods of Preparing Administrable Compositions Containing the Rapamycin Analogues

In one aspect, the present methods include administration of a pharmaceutical composition containing one or more rapamycin analogues of formula I. As used herein, reference to compositions containing “a rapamycin analogue” or “the rapamycin analogue” are intended to encompass compositions containing one or more rapamycin analogues of formula I. The composition can be administered to a mammalian subject by several different routes including solid or liquid form.

Solid forms, including tablets, capsules, and caplets, containing the rapamycin analogue can be formed by blending the rapamycin analogue with one or more of the components described above. In one embodiment, the components of the composition are dry or wet blended. In another embodiment, the components are dry granulated. In a further embodiment, the components are suspended or dissolved in a liquid and added to a form suitable for administration to a mammalian subject.

Liquid forms containing the rapamycin analogue can be formed by dissolving or suspending the rapamycin analogue in a liquid suitable for administration to a mammalian subject.

Compositions containing the rapamycin analogue of formula I can be prepared by combining the rapamycin analogue and a pharmaceutically acceptable carrier.

The compositions described herein containing the rapamycin analogue can be formulated in any form suitable for the desired route of delivery using a pharmaceutically effective amount of the rapamycin analogue. For example, the compositions of the invention can be delivered by a route such as oral, dermal, transdermal, intrabronchial, intranasal, intravenous, intramuscular, subcutaneous, parenteral, intraperitoneal, intranasal, vaginal, rectal, sublingual, intracranial, epidural, intratracheal, or by sustained release.

The oral dosage tablet composition of this invention can also be used to make oral dosage tablets containing derivatives of the rapamycin analogue, including, but not limited to, esters, carbamates, sulfates, ethers, oximes, carbonates, and the like which are known to those of skill in the art.

A pharmaceutically effective amount of the rapamycin analogue can vary depending on the compound(s), mode of delivery, severity of the condition being treated, and any other active ingredients used in the composition. The dosing regimen can also be adjusted to provide the optimal therapeutic response. Several divided doses can be delivered daily, e.g., in divided doses 2 to 4 times a day, or a single dose can be delivered. The dose can however be proportionally reduced or increased as indicated by the exigencies of the therapeutic situation. In one embodiment, the delivery is on a daily, weekly, or monthly basis. In another embodiment, the delivery is on a daily delivery. However, daily dosages can be lowered or raised based on the periodic delivery.

The rapamycin analogues can be combined with one or more pharmaceutically acceptable carriers or excipients including, without limitation, solid and liquid carriers, which are compatible with the compositions of the present invention. Such carriers include adjuvants, syrups, elixirs, diluents, binders, lubricants, surfactants, granulating agents, disintegrating agents, emollients, metal chelators, pH adjustors, surfactants, fillers, disintegrants, and combinations thereof, among others. In one embodiment, the rapamycin analogue is combined with metal chelators, pH adjustors, surfactants, fillers, disintegrants, lubricants, and binders.

Adjuvants can include, without limitation, flavoring agents, coloring agents, preservatives, and supplemental antioxidants, which can include vitamin E, ascorbic acid, butylated hydroxytoluene (BHT) and butylated hydroxyanisole (BHA).

Binders can include, without limitation, cellulose, methylcellulose, hydroxymethylcellulose, carboxymethylcellulose calcium, carboxymethylcellulose sodium, hydroxypropylcellulose, hydroxypropylmethylcellulose phthalate, microcrystalline cellulose, noncrystalline cellulose, polypropylpyrrolidone, polyvinylpyrrolidone (povidone, PVP), gelatin, gum arabic and acacia, polyethylene glycols, starch, sugars such as sucrose, kaolin, dextrose, and lactose, cholesterol, tragacanth, stearic acid, gelatin, casein, lecithin (phosphatides), cetostearyl alcohol, cetyl alcohol, cetyl esters wax, dextrates, dextrin, glyceryl monooleate, glyceryl monostearate, glyceryl palmitostearate, polyoxyethylene alkyl ethers, polyoxyethylene castor oil derivatives, polyoxyethylene stearates, polyvinyl alcohol, and gelatin, among others. In one embodiment, the binder is povidone, hydroxypropylmethylcellulose, carboxymethylcellulose, or gelatin. In another embodiment, the binder is povidone.

Lubricants can include magnesium stearate, light anhydrous silicic acid, talc, stearic acid, sodium lauryl sulfate, and sodium stearyl furamate, among others. In one embodiment, the lubricant is magnesium stearate, stearic acid, or sodium stearyl furamate. In another embodiment, the lubricant is magnesium stearate.

Granulating agents can include, without limitation, silicon dioxide, microcrystalline cellulose, starch, calcium carbonate, pectin, crospovidone, and polyplasdone, among others.

Disintegrating agents or disintegrants can include croscarmellose sodium, starch, carboxymethylcellulose, substituted hydroxypropylcellulose, sodium bicarbonate, calcium phosphate, calcium citrate, sodium starch glycolate, pregelatinized starch or crospovidone, among others. In one embodiment, the disintegrant is croscarmellose sodium.

Emollients can include, without limitation, stearyl alcohol, mink oil, cetyl alcohol, oleyl alcohol, isopropyl laurate, polyethylene glycol, olive oil, petroleum jelly, palmitic acid, oleic acid, and myristyl myristate.

Surfactants can include polysorbates, sorbitan esters, poloxamer, or sodium lauryl sulfate. In one embodiment, the surfactant is sodium lauryl sulfate.

Metal chelators can include physiologically acceptable chelating agents including edetic acid, malic acid, or fumaric acid. In one embodiment, the metal chelator is edetic acid.

pH adjusters can also be utilized to adjust the pH of a solution containing the rapamycin analogue to about 4 to about 6. In one embodiment, the pH of a solution containing the rapamycin analogue is adjusted to a pH of about 4.6. pH adjustors can include physiologically acceptable agents including citric acid, ascorbic acid, fumaric acid, or malic acid, and salts thereof. In one embodiment, the pH adjuster is citric acid.

Fillers that can be used according to the present invention include anhydrous lactose, microcrystalline cellulose, mannitol, calcium phosphate, pregelatinized starch, or sucrose. In one embodiment, the filler is anhydrous lactose. In another embodiment, the filler is microcrystalline cellulose.

In one embodiment, compositions containing the rapamycin analogue of formula I are delivered orally by tablet, caplet or capsule, microcapsules, dispersible powder, granule, suspension, syrup, elixir, and aerosol. Desirably, when compositions containing the rapamycin analogue are delivered orally, delivery is by tablets and hard- or liquid-filled capsules.

In another embodiment, the compositions containing the rapamycin analogue can be delivered intravenously, intramuscularly, subcutaneously, parenterally and intraperitoneally in the form of sterile injectable solutions, suspensions, dispersions, and powders which are fluid to the extent that easy syringe ability exits. Such injectable compositions are sterile and stable under conditions of manufacture and storage, and free of the contaminating action of microorganisms such as bacteria and fungi.

In some embodiments of the present methods, the administration of the compound of formula I, or composition comprising the compound of formula I, is performed by intravenous administration. For example, such a pharmaceutical composition can contain one or more rapamycin analogues of formula I, and also comprise sterile isotonic aqueous buffer. Where necessary, the compositions can also include a solubilizing agent. Compositions for intravenous administration can also optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the compound or a pharmaceutically acceptable salt of the compound of formula I is to be administered by infusion, it can be dispensed, for example, with an infusion bottle containing sterile pharmaceutical grade water or saline. Where the compound or a pharmaceutically acceptable salt of the compound is administered by injection, an ampule of sterile water for injection or saline can be provided so that the ingredients can be mixed prior to administration.

In a further embodiment, compositions containing the rapamycin analogue can be delivered rectally in the form of a conventional suppository.

In another embodiment, compositions containing the rapamycin analogue can be delivered vaginally in the form of a conventional suppository, cream, gel, ring, or coated intrauterine device (IUD).

In another embodiment, compositions containing the rapamycin analogue can be delivered via coating or impregnating of a supporting structure, i.e., a framework capable of containing of supporting pharmaceutically acceptable carrier or excipient containing a compound of the invention, e.g., vascular stents or shunts, coronary stents, peripheral stents, catheters, arterio-venous grafts, by-pass grafts, and drug delivery balloons for use in the vasculature. In one embodiment, coatings suitable for use include, but are not limited to, polymeric coatings composed of any polymeric material in which the compound of the invention is substantially soluble. Supporting structures and coating or impregnating methods, e.g., those described in U.S. Pat. No. 6,890,546, are known to those of skill in the art and are not a limitation of the present invention.

In yet another embodiment, compositions containing the rapamycin analogue can be delivered intranasally or intrabronchially in the form of an aerosol.

The rapamycin analogues are administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions are advantageously included, such as flavoring agents, coloring agents, preserving agents, and antioxidants, for example, vitamin E, ascorbic acid, BHT and BHA.

Pharmaceutical compositions from the standpoint of ease of preparation and administration are solid compositions, such as, tablets and hard-filled or liquid-filled capsules. The rapamycin analogues are also administered parenterally or intraperitoneally. Solutions or suspensions of these active compounds as a free base or pharmacologically acceptable salt are prepared in water suitably mixed with a surfactant such as hydroxypropylcellulose. Dispersions are also prepared in glycerol, liquid, polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form is sterile and fluid to the extent that easy syringe ability exits. It is stable under conditions of manufacture and storage and is preserved against the contaminating action of microorganisms such as bacterial and fungi. The carrier is a solvent or dispersion medium containing, for example, water, ethanol (e.g., glycerol, propylene glycol and liquid polyethylene glycol), suitable mixtures thereof, and vegetable oil.

### V. Kits of the Invention

The present invention also provides kits or packages containing the rapamycin analogues. Kits can include the rapamycin analogue and a carrier suitable for administration to a mammalian subject as discussed above. The kits can also contain the reagents required to prepare the rapamycin analogues and include a rapamycin, an optionally substituted nitrosobenzene, and a solvent.

The kits can optionally include other reagents to form other rapamycin analogues and include hydrogenation agents.

The kit can further contain instructions for performing the methods of the present invention. Also provided in a kit can be other suitable chemicals, disposable gloves, decontamination instructions, applicator sticks or containers, and sample preparator cups.

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

### EXAMPLES

### Example 1

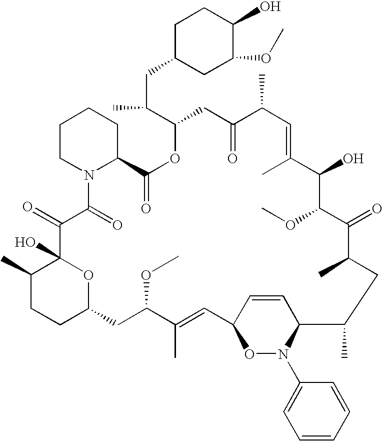

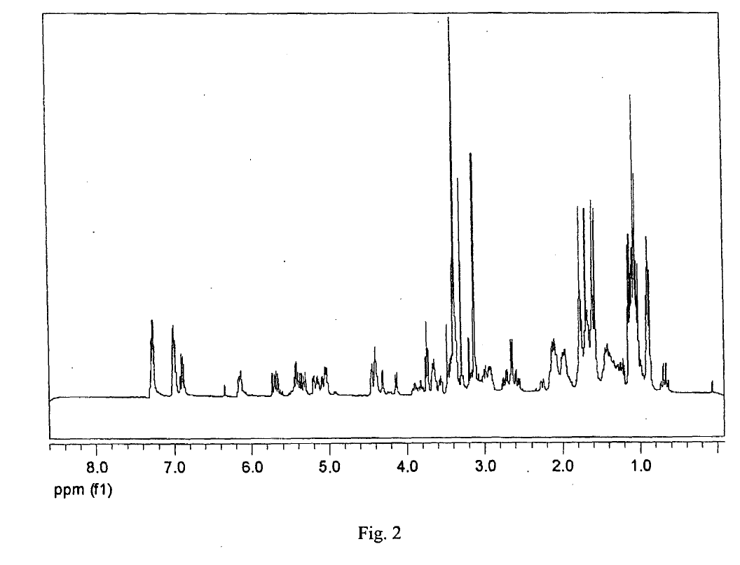

Table 1 provides the mass spectral (MS) data and FIG. 2 provides the nuclear magnetic resonance (NMR) spectrum for the compound produced by the following two alternative routes.

TABLE 1
Theoretical Neutral Mass: 1020.59226
Exact Mass High Resolution Results

| Adduct | Exptl. | Exact | Mmu | ppm | RI % |
|---|---|---|---|---|---|
| [M + H]¹⁺ | 1021.59780 | 1021.59954 | −1.74 | −1.70 | 100.0 |
| [M + Na]¹⁺ | 1043.57740 | 1043.58148 | −4.08 | −3.91 | 17.8 |
| [M + NH₄]¹⁺ | 1038.62305 | 1038.62608 | −3.03 | −2.92 | 1.2 |
| [M + 2H]²⁺ | 511.30109 | 511.30341 | −2.32 | −4.53 | 1.8 |
| [M + CH₃OH + H]¹⁺ | 1053.62596 | 1053.62575 | 0.21 | 0.20 | 1.5 |

A. Route 1

Rapamycin (2.5 gram (g), 2.73 millimolar (mmol)) was dissolved in 200 milliliter (mL) p-dioxane. To this solution was added, dropwise, a solution of nitrosobenzene (1.50 g, 5 equivalents (eq) in 200 mL p-dioxane. The reaction mixture was stirred at 50° C. for 64 hours, and then the products were chromatographed via reversed-phase high performance liquid chromatography (HPLC) (column: 200×50 mm YMC ODS-A, mobile phase: 80% methanol:water, ramped flow rate from 10 mL/min to 35 mL/min in 10 minutes, then hold at 35 mL/min for an additional 65 minutes) to yield 1.22 g of the product (44% yield, t_{R}=12.1 min, analytical HPLC conditions: column=YMC ODS-A S-3 120 Å, mobile phase/gradient: 95% water (+0.025% formic acid)/acetonitrile (+0.025% formic acid) to 5% water in 6 minutes, hold at 5% for 9 minutes, flow=0.30 mL/min).

B. Route 2—Alternate Route

Rapamycin (0.3 g, 0.328 mmol) was dissolved in 5 mL toluene with gentle heating. To this solution was added, dropwise, a solution of nitrosobenzene (0.1 g, 3 eq) in 5 mL toluene. The reaction mixture was stirred at 70° C. for 16 hours, and then the products were chromatographed via reversed-phase high performance liquid chromatography (HPLC) (column: 250×20 mm YMC ODS-A with 50×20 guard, mobile phase: 80% to 85% methanol:water in 40 minutes, flow=20 mL/min) to yield 0.139 g of the product (42% yield, t_{R}=12.1 min., analytical HPLC conditions: column=YMC ODS-A S-3 120 Å, mobile phase/gradient: 95% water (+0.025% formic acid)/acetonitrile (+0.025% formic acid) to 5% water in 6 minutes, hold at 5% for 9 minutes, flow=0.30 mL/min.).

### Example 2

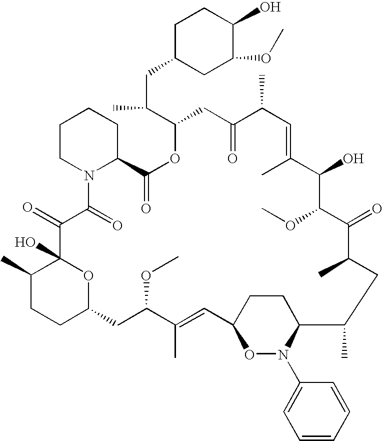

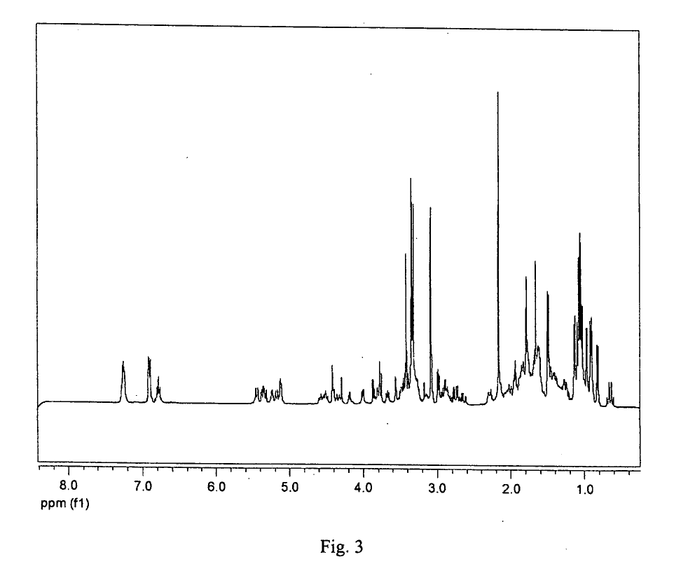

Table 2 provides the mass spectral (MS) data and FIG. 3 provides the nuclear magnetic resonance (NMR) spectrum for the compound produced by the following two alternative routes.

A. Route 1

The compound prepared according to Example 1 (0.29 g, 0.284 mmol) was dissolved in 7 mL methanol in an 18 millimeter (mm) test-tube, and a spatula tip of Pd/C catalyst (Aldrich) was added. The mixture was hydrogenated on a Parr apparatus for 15 minutes at 2.0 atmosphere H₂. The products were chromatographed via reversed-phase HPLC (column: 250×20 mm YMC ODS-A with 50×20 guard, mobile phase: 80% methanol:water for 15 minutes, then to 85% in 5 minutes, then held at 85% for 20 minutes, flow=20 mL/min.) to yield 0.089 g of the product (31% yield, t_{R}=12.6 min., analytical HPLC conditions: column=YMC ODS-A S-3 120 Å, mobile phase/gradient: 95% water (+0.025% formic acid)/acetonitrile (+0.025% formic acid) to 5% water in 6 minutes, hold at 5% for 9 minutes, flow=0.30 mL/min.)

B. Route 2—Alternate Route

The compound prepared according to Example 1 (9.85 g, 9.65 mmol) was dissolved in 50 mL methanol, and 3 spatula tips of Pd/C catalyst (Aldrich) was added. The mixture was hydrogenated on a Parr apparatus for 2.5 hours at 2.5 atmospheres H₂. The products were chromatographed via reversed-phase HPLC (column: 250×50 mm YMC ODS-A, mobile phase: 80% methanol:water for 40 minutes, then to 85% in 5 minutes, then held at 85% for 35 minutes, flow=35 mL/min.) to yield 3.35 g of the product (15% yield, t_{R}=12.2 min., analytical HPLC conditions: column=YMC ODS-A S-3 120 Å, mobile phase/gradient: 95% water (+0.025% formic acid)/acetonitrile (+0.025% formic acid) to 5% water in 6 minutes, hold at 5% for 9 minutes, flow=0.30 mL/min.)

TABLE 2
Theoretical Neutral Mass: 1022.60791
Exact Mass High Resolution Results

| Adduct | Exptl. | Exact | mmu | ppm | RI % |
|---|---|---|---|---|---|
| [M + H]¹⁺ | 1023.61722 | 1023.61519 | 2.03 | 1.99 | 100.0 |
| [M + Na]¹⁺ | 1045.59943 | 1045.59713 | 2.30 | 2.20 | 10.5 |

### Example 3

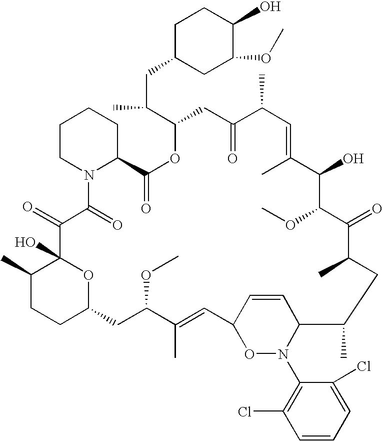

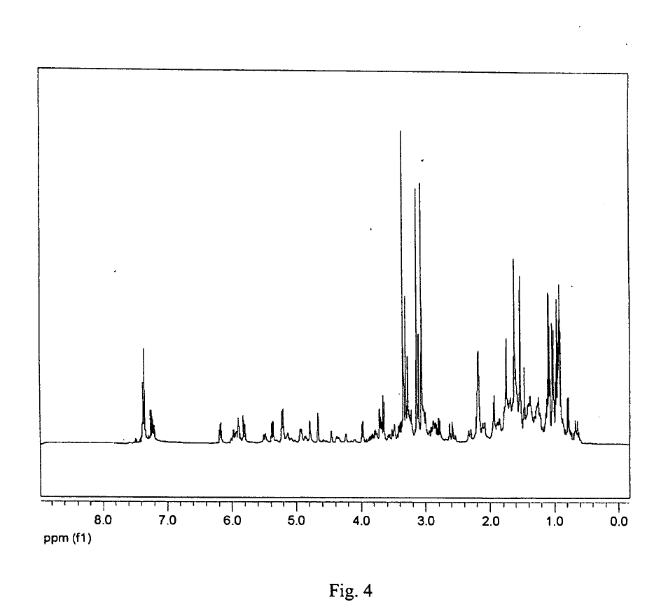

Rapamycin (0.25 g, 0.274 mmol) was dissolved in 5° C. mL toluene with gentle heating. To this solution was added, dropwise, a solution of 2,6-dichloronitrosobenzene (0.144 g, 3 eq) in 7 mL toluene. The reaction mixture was stirred at 80° C. for 36 hours, and then the products were chromatographed via reversed-phase HPLC (column: 250×20 mm YMC ODS-A with 50×20 guard, mobile phase: 80% to 85% methanol:water in 40 minutes, flow=20 mL/min.) to yield 0.046 g of the product (15% yield, t_{R}=13.0 minutes, analytical HPLC conditions: column=YMC ODS-A S-3 120 Å, mobile phase/gradient: 95% water (+0.025% formic acid)/acetonitrile (+0.025% formic acid) to 5% water in 6 minutes, hold at 5% for 9 minutes, flow=0.30 mL/min.). The MS data is provided in Table 3 and FIG. 4 provides the NMR spectrum.

TABLE 3
Theoretical Neutral Mass: 1088.51431
Exact Mass High Resolution Results

| Adduct | Exptl. | Exact | mmu | ppm | RI % |
|---|---|---|---|---|---|
| [M + H]¹⁺ | 1089.52125 | 1089.52159 | −0.34 | −0.31 | 19.1 |
| [M + Na]¹⁺ | 1111.50044 | 1111.50353 | −3.09 | −2.78 | 18.1 |
| [M + NH₄]¹⁺ | 1106.54443 | 1106.54813 | −3.70 | −3.35 | 1.2 |

### Example 4

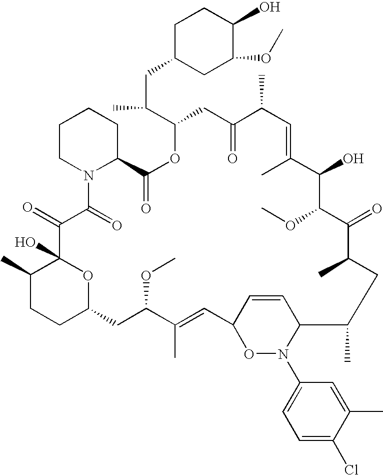

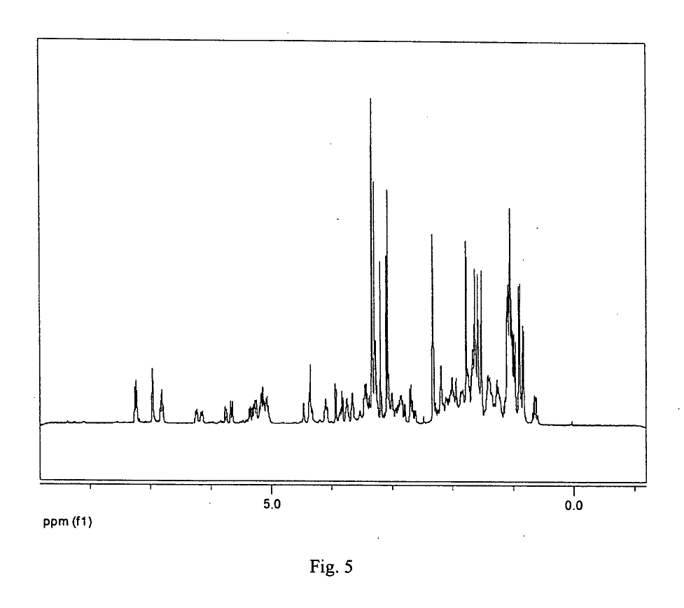

The synthesis of this example was performed as described in Example 3 and employing 0.05 g of rapamycin and 0.042 g of 1-chloro-2-methyl-4-nitrosobenzene to give 0.012 g of the product (20% yield, t_{R}=12.8 min.). The MS data is provided in Table 4 and FIG. 5 provides the NMR spectrum.

TABLE 4

| Experimental | Elemental Formula | Predicted | Δ | Δ | Ion Assignment |
|---|---|---|---|---|---|
| Mass | (proposed) | Mass | (mmu) | (ppm) | (proposed) |
| 1091.55815 | C₅₈H₈₅ClN₂O₁₄Na¹⁺ | 1091.55815 | 0.00 | 0.00 | [M + Na]¹⁺ |
| 936.54361 | C₅₁H₇₉NO₁₃Na¹⁺ | 936.54436 | −0.75 | −0.80 | [M + Na]¹⁺ —C₇H₆ClNO |

### Example 5

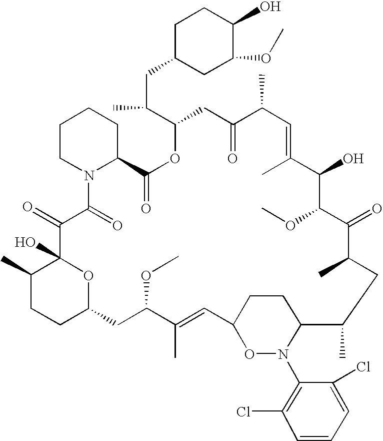

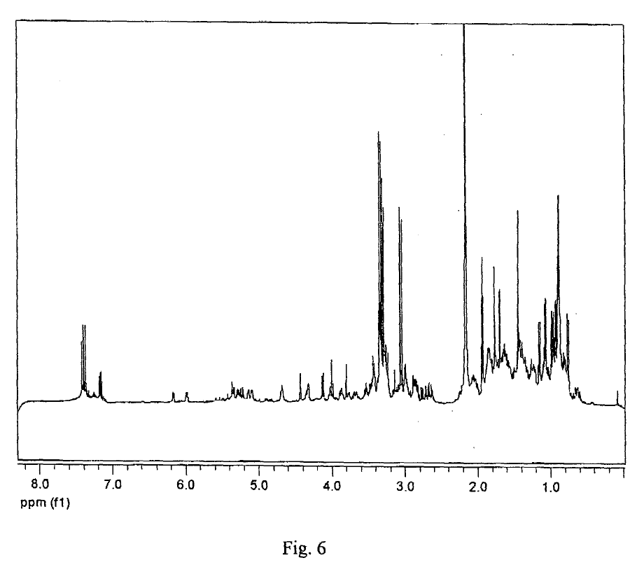

The product from Example 3 (0.046 g, 0.0422 mmol) was dissolved in 5 mL methanol in an 18 mm test-tube, and a spatula tip of Pd/C catalyst (Aldrich) was added. The mixture was hydrogenated on a Parr apparatus for 60 minutes at 3 atmospheres H₂. The products were chromatographed via reversed-phase HPLC (column: 250×20 mm YMC ODS-A with 50×20 guard, mobile phase: 85% methanol:water to 90% in 15 minutes, then hold at 90% for 25 minutes, flow=20 mL/min.) to yield 0.005 g of the product (11% yield, t_{R}=10.0 minutes, analytical HPLC conditions: column=YMC ODS-A S-3 120 Å, mobile phase/gradient: 95% water (+0.025% formic acid)/acetonitrile (+0.025% formic acid) to 5% water in 6 minutes, hold at 5% for 9 minutes, flow=0.30 mL/min.). The MS data is provided in Table 5 and FIG. 6 provides the NMR spectrum.

TABLE 5
Theoretical Neutral Mass: 1090.52996
Exact Mass High Resolution Results

| Adduct | Exptl. | Exact | mmu | ppm | RI % |
|---|---|---|---|---|---|
| [M + H]¹⁺ | 1091.53497 | 1091.53724 | −2.27 | −2.08 | 4.3 |
| [M + Na]¹⁺ | 1113.52166 | 1113.51918 | 2.48 | 2.23 | 4.8 |
| [M + NH₄]¹⁺ | 1108.56627 | 1108.56378 | 2.49 | 2.24 | 15.5 |

### Example 6

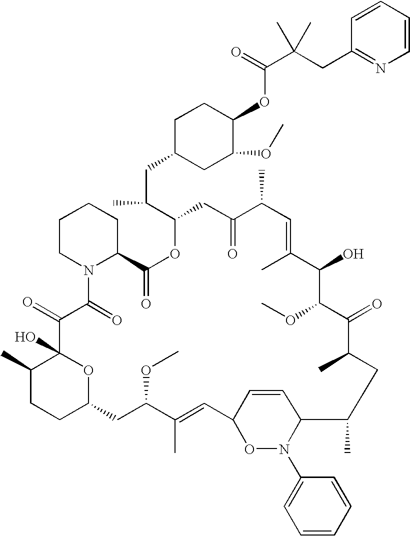

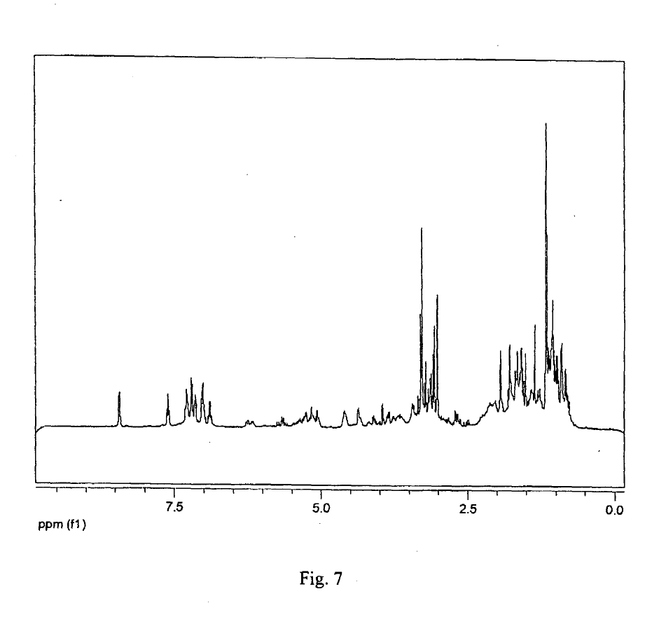

The synthesis of this example was performed as described in Example 3 and using 0.108 g (0.099 mmol) rapamycin 42-ester with 2,2-dimethyl-3-(pyridine-2-yl)-propionic acid (prepared according to the method of U.S. Pat. No. 5,385,908) and 0.032 g nitrosobenzene (0.297 mmol, 3 eq.). The reaction was stirred at 70° C. for 40 hours and then the products were chromatographed via reversed-phase HPLC (column: 250×20 mm YMC ODS-A with 50×20 guard, mobile phase: 80% to 85% methanol:water in 15 minutes, then to 90% methanol in 10 minutes, then hold at 90% for 15 minutes, flow=20 mL/min.) to yield 0.007 g of the product (6% yield, t_{R}=13.0 minutes). The MS data is shown in Table 6 and FIG. 7 provides the NMR spectrum.

TABLE 6
Theoretical Neutral Mass: 1181.67632
Exact Mass High Resolution Results

| Adduct | Exptl. | Exact | mmu | ppm | RI % |
|---|---|---|---|---|---|
| [M + H]¹⁺ | 1182.68360 | 1182.68747 | 3.87 | 3.28 | 23.2 |
| [M + 2H]²⁺ | 591.84544 | 591.84715 | 1.71 | 2.90 | 79.6 |
| [M + H + Na]²⁺ | 602.83641 | 602.83743 | 1.02 | 1.70 | 34.1 |

### Example 7

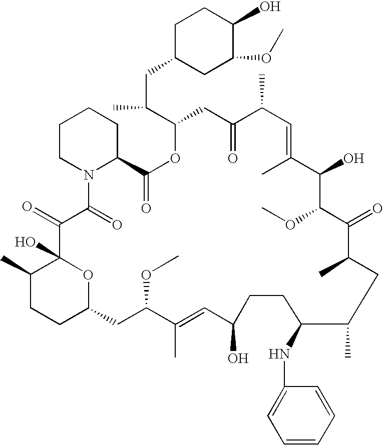

The compound prepared according to Example 1 (0.031 g, 0.03 mmol) was dissolved in 5 mL methanol in an 18 mm test-tube, and a spatula tip of Pd/C catalyst (Aldrich) was added. The mixture was hydrogenated on a Parr apparatus for 30 minutes at 2.0 atmosphere H₂. The products were chromatographed via reversed-phase HPLC (column: 250×20 mm YMC ODS-A with 50×20 guard, mobile phase: 80% methanol:water for 15 minutes, then to 85% in 5 minutes, then held at 85% for 20 minutes, flow=20 mL/min.) to yield 0.016 g of the product (55% yield, t_{R}=9.95 min., analytical HPLC conditions: column=YMC ODS-A S-3 120 Å, mobile phase/gradient: 95% water (+0.025% formic acid)/acetonitrile (+0.025% formic acid) to 5% water in 6 minutes, hold at 5% for 9 minutes, flow=0.30 mL/min.) The MS data is provided in Table 7:

TABLE 7
Exact Mass High Resolution Results

| Adduct | Exptl. | Exact | mmu | ppm | RI % |
|---|---|---|---|---|---|
| [M + H]1+ | 1025.62887 | 1025.63084 | −1.97 | −1.92 | 38.0 |

### Example 8

Mesencephalic dopaminergic neuron cultures were prepared as described in Pong et al., J. Neurochem. 69: 986-994, 1997, which is incorporated herein by reference in its entirety. Embryonic day 15 (E15) rat fetuses were collected and dissected in ice-cold phosphate-buffered saline (PBS). The ventral piece of tissue compromising the mesencephalic dopaminergic region was dissected out. Dissected pieces of tissue were pooled together and transferred to an enzymatic dissociation medium containing 20 IU/mL papain in Earle's balanced salt solution (Worthington Biochemical, Freehold, N.J., USA) and incubated for 60 minutes at 37° C. After enzymatic dissociation, the papain solution was aspirated and the tissue mechanically triturated with a fire-polished glass Pasteur pipette in complete medium (equal volumes of minimum essential medium (MEM) and F-12 nutrient mixture (GibcoBRL) supplemented with 0.1 mg/mL apotransferrin and 2.5 μg/mL insulin) containing 2,000 IU/mL DNase and 10 mg/mL ovomucoid protease inhibitor.

For dopamine uptake experiments, single-cell suspensions in complete media were seeded on poly-L-ornithine and laminin coated 24-well plates. The cultures were maintained for seven days prior to experimentation. Cultures were pretreated with various concentrations of the compound for 24 hours, then exposed to 10 mM MPP+ for 1 hour. Following the 1 hour incubation, media was exchanged three times and fresh compound was added for an additional 48 hours.

After 48 hours growth of mesencephalic dopaminergic neuron cultures following MPP+ exposure, high-affinity 3H-dopamine uptake was performed using a modified method described by Prochiantz et al., Nature 293: 570-572, 1981, which is incorporated herein by reference. Cultures were washed with pre-warmed PBS containing 5.6 mM glucose and 1 mM ascorbic acid. Cultures were then incubated for 15 minutes at 37° C. with 50 nM 3H-dopamine (31 Ci/mmol, DuPont-NEN, Wilmington, Del., USA). The cultures were washed twice with buffer and lysed with 0.5N NaOH. The lysate was transferred to a scintillation vial containing Ultima Gold scintillation cocktail and radioactivity was determined with a liquid scintillation counter. Alternatively, culture lysates can be washed twice with buffer, incubated for 2 hours at room temperature with Optiphase Supermix scintillation cocktail (Wallac Scintillation Products, Gaithersburg, Md., USA), and radioactivity measured with a liquid scintillation counter.

Dissociated cortical neuron cultures were prepared as previously described (Liu et al., Screening of Immunophilin Ligands by Quantitative Analysis of Neurofilament Expression and Neurite Outgrowth in Cultured Neurons and Cells, J. Neurosci. Methods, 2007 Jul. 30; 163(2):310-20).

Briefly, embryonic day 15 rat fetuses were collected and dissected in ice-cold PBS. Dissected cortices were pooled together and transferred to an enzymatic dissociation medium containing papain. After 30 minutes, the tissue was mechanically triturated with a fire-polished glass Pasteur pipette. Single-cell suspensions in complete media were seeded on poly-L-ornithine and laminin coated 96-well plates. After 24 hours, cultures were treated with various concentrations of compound for 72 hours. The cultures were then fixed and stained with an anti-tubulin primary antibody (TUJ-1) and a fluorescent-tagged secondary antibody. Neurite outgrowth was determined by using the Enhanced Neurite Outgrowth (ENO) algorithm with the Cellomics ArrayScan and expressed as average neurite length or total neurite length per cell.

Spinal cord neuron cultures were prepared from embryonic day 15 (E15) rat embryos (Liu et al., Screening of Immunophilin Ligands by Quantitative Analysis of Neurofilament Expression and Neurite Outgrowth in Cultured Neurons and Cells, J. Neurosci. Methods, 2007 Jul. 30; 163(2):310-20). The embryos were collected and their spinal cords were removed in ice-cold phosphate-buffered saline (PBS) without Ca²⁺ and Mg²⁺. Dissected pieces of spinal cord tissue were pooled together and transferred to an enzymatic dissociation media containing 20 IU/mL papain in Earle's balanced salt solution (Worthington Biochemical, Freehold, N.J.) and incubated for 30 minutes at 37° C. After enzymatic dissociation, the papain solution was aspirated and the tissue mechanically triturated with a fire-polished Pasteur pipette in complete media [Neurobasal Medium with B-27 supplement (Gibco, Grand Island, N.Y.), 100 IU/mL penicillin, 100 g/mL streptomycin, 3.3 g/mL aphidicolin, 0.5 mM glutamate] containing 2,000 IU/mL DNase and 10 mg/mL ovomucoid protease inhibitor. Single-cell suspensions in complete media were plated on pre-coated poly-L-ornithine/laminin 96-well plates (Becton-Dickinson, Bedford, Mass.) at a density of 1.0×10⁴ cells/well. Spinal cord neurons were maintained for 24 hours then exposed to vehicle or various concentrations of compound for 72 hours.

The compounds of Examples 1-3 were all active in cortical neuron assays with an EC₅₀ less than 1 μM. The compounds of Examples 1 and 6 were all active in dopaminergic uptake assays with an EC₅₀ less than 1 μM. The compounds of Examples 1-3 and 6 were all active in spinal cord neuron assays with an EC₅₀ less than 1 μM

In comparison, CCI-779 and rapamycin were considered inactive in cortical neuron assays and dopaminergic uptake assays with EC₅₀ values of greater than 1 μM. Rapamycin phenyltriazolinedione was active in the dopaminergic uptake assay with an EC₅₀ value of less than 1 μM.

### In Vivo Pharmacology

The compound of Example 2 was evaluated in two known rodent models of ischemic stroke. [Bederson J B, Pitts L H, Tsuji M, Nishimura M C, Davis R L, Bartkowski H (1986) Rat middle cerebral artery occlusion: evaluation of the model and development of a neurologic examination. Stroke 17(3): 472-476; Chen S T, Hsu C Y, Hogan E L, Maricq H Balentine J D (1986) A model of focal ischemic stroke in the rat: reproducible extensive cortical infarction. Stroke 17:738-743. DeRyck M, van Reempts J, Duytschaever H, van Deuren B, Clincke G (1992) Neocortical localization of tactile/proprioceptive limb placing reactions in the rat. Brain Res 573:44-60; Longa Z E, Weinstein P R, Carlson S, Cummins R. (1989) Reversible middle cerebral artery occlusion without craniectomy in rats. Stroke 20:294-297.]

### Example 9

### Transient Middle Cerebral Artery Occlusion (tmCAO) Model

The first model is a transient middle cerebral artery occlusion (tMCAO), in which the MCA is occluded for 90 minutes, followed by 72 hours of reperfusion. Rats were subjected to tMCAO and treated with either the compound of Example 2 (3 mg/kg, 10 mg/kg, or 30 mg/kg) via intravenous delivery [iv vehicle: 5% Tween 80, 5% PEG 400, 4% ethanol] or vehicle alone at 4, 6, 24, and 48 hours post-occlusion (4 total doses). Neurological deficits (modified Bederson scale) and weight loss were monitored at 0, 24, 48, and 72 hours post occlusion. At the end of the evaluation period, rats were euthanized and infarct volume was determined. A modified Bederson scale was used to evaluate neurological deficits following stroke. Animals were scored on a scale of 0 to 5, based on five parameters (forelimb flexion, torso twisting, resistant to lateral push, circling to contralateral side, and spontaneous circling). A score of 0 is normal whereas a score of 5 represents severe neurological deficits.

Neurological Deficits

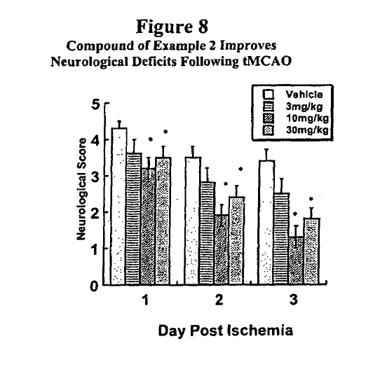

1. 10 mg/kg or 30 mg/kg of the compound of Example 2 significantly improved neurological deficits following tMCAO when compared to vehicle treated animals. 3 mg/kg of the compound of Example 2 showed trends of improving neurological deficits, but did not reach statistical significance when compared to vehicle treatment (FIG. 8).

2. Weight Loss

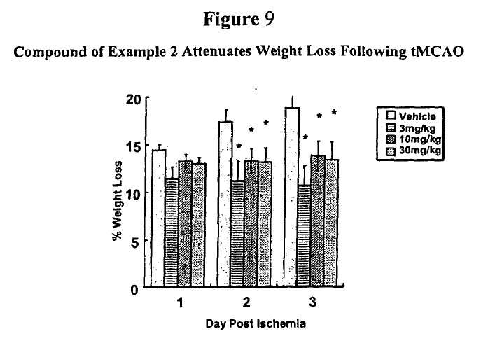

Following tMCAO, animals tend to lose a significant amount of body weight, due to their poor overall health. The compound of Example 2 (3 mg/kg, 10 mg kg, and 30 mg/kg) significantly attenuated weight loss following tMCAO (FIG. 9), suggesting that the animals were healthier overall.

3. Infarct Volume

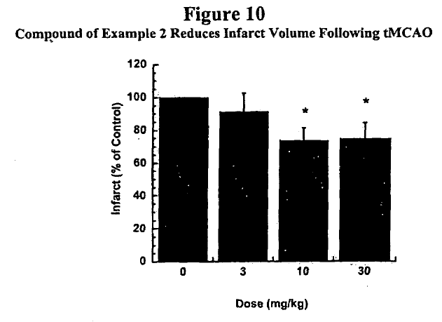

At the end of the 72 hour evaluation period, animals were euthanized and infarct volume (dead tissue) was determined. 10 mg/kg and 30 mg/kg of the compound of Example 2 significantly reduced infarct volume by 24% and 23%, respectively. Although 3 mg/kg of the compound of Example 2 reduced infarct volume by 9%, this reduction did not reach statistical significance when compared to vehicle treatment (FIG. 10).

### Example 10

### Permanent Occlusion of Middle Cerebral Artery (pMCAO)

Administration of Compound of Example 2—10 mg/kg i.v. 1.5, 5.5, 24, 48, and 72 hours post ischemia.

Adult male Wistar rats (Charles River, Wilmington, Mass.) 270 g to 300 g were anesthetized with 3% isoflurane in 70% nitrous oxide and 30% oxygen through a nose cone. Temperature was maintained at 37° C. throughout the surgery using a heating lamp. Permanent occlusion of MCAO was induced by electro cauterization of the distal portion of the MCA (via a craniotomy) with a 90 minute ligation of both carotid arteries to interrupt collateral circulation (Chen S T, Hsu C Y, Hogan E L, Maricq H Balentine J D (1986) A model of focal ischemic stroke in the rat: reproducible extensive cortical infarction. Stroke 17:738-743). Compound was administered 10 mg/kg i.v. 1.5, 5.5, 24, 48, and 72 hours post ischemia. Rats were kept for 21 days for long-term functional recovery evaluation. Three behavioral tests, modified from earlier tests reported by Bederson et al., (Bederson J B, Pitts L H, Tsuji M, Nishimura M C, Davis R L, Bartkowski H (1986) Rat middle cerebral artery occlusion: evaluation of the model and development of a neurologic examination. Stroke 17(3): 472-476) and DeRyck et al. (DeRyck M, van Reempts J, Duytschaever H, van Deuren B, Clincke G (1992) Neocortical localization of tactile/proprioceptive limb placing reactions in the rat. Brain Res 573:44-60), were used to assess sensorimotor and reflex function. Briefly, for the postural test, rats were suspended from the tail approximately 30 cm above the bench top. Rats extending both forelimbs toward the table were scored as 0, flexing contralateral limb toward the body and/or rotating the contralateral shoulder and limb medially were scored as 1, and rolling up the body toward the contralateral side and attempting to grasp the tail were scored as 2. The forelimb placement test is comprised of two subtests, visual and tactile placing test. For visual placing test, rats were held with forelimbs hanging free and were brought close either from front or sideway to a tabletop. For tactile placing test, the rats were held so that it cannot see the tabletop. The dorsal and lateral surface of the forepaw touched lightly to the tabletop. For each test, scoring was, 0 if the placing response was immediate and normal; 1 if the placing was delayed (>2 seconds) or occasional; 2 if there was no response. For hind limb placement test, rats were held on the edge of the bench top and the contralateral hind limb was pulled off the edge and released. Rats retracting hind limb back on bench top immediately were scored as 0, delaying (>2 seconds) were scored as 1, and unable to retract hind limb were score as 2. Total Score was ranged from 0 to 12. The compound prepared in Example 2 showed statistically significant reduction of behavior deficit scoring in rats after I.V. administration (10 mg/kg) following pMCAO.

### Example 11

### Further pMCAO Studies—Treatment Paradigms for Determination of Clinical Relevance

Rats were subjected to pMCAO and treated with either the compound of Example 2 delivered intravenously into rat tail vein [iv vehicle: 5% Tween 80, 5% PEG 400, 4% ethanol] or vehicle alone. Three different treatment paradigms were used to determine the clinical relevance of the compound of Example 2. Long-term sensorimotor deficits are monitored (tactile/proprioceptive/hindlimb placement tests) over a 3-week and 6-month period. The animals are scored on a scale of 0-12 (normal to severe neurological deficits).

1. First pMCAO Study

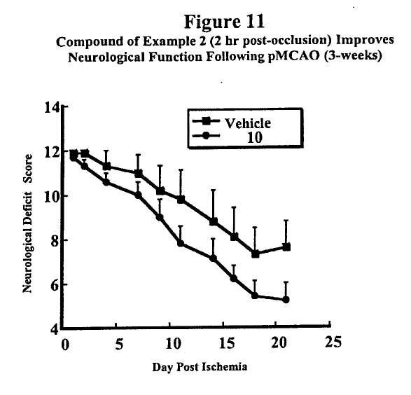

In the first study, the first dose of 10 mg/kg of the compound of Example 2 is given 2 hours post-occlusion, followed by 4 additional doses, 24 hours apart (5 total doses). Neurological deficits were monitored over a 3-week period. 10 mg/kg of the compound of Example 2 significantly improved and accelerated neurological function when compared to vehicle treatment (FIG. 11).

2. Second Study of pMCAO

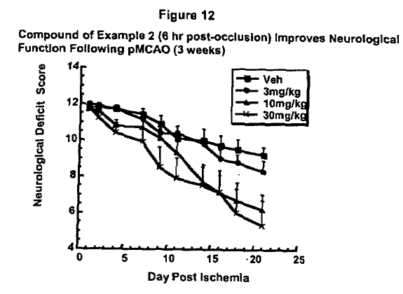

In the second study, the first dose of the compound of Example 2 is given 6 hours post-occlusion, followed by 4 additional doses, 24 hours apart (5 total doses). Neurological deficits were monitored over a 3-week period. 10 mg/kg and 30 mg/kg of the compound of Example 2 significantly improved and accelerated neurological function when compared to vehicle treatment. 3 mg/kg of the compound of Example 2 did not improve neurological function (FIG. 12).

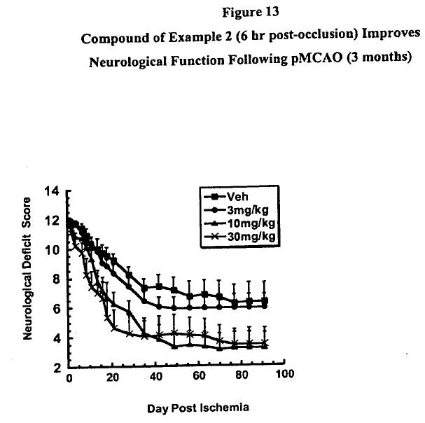

These animals were maintained for an additional 3 months. During this period, neurological deficits were continually monitored, to determine whether the effect seen at 21 days is stable and long lasting. 10 mg/kg and 30 mg/kg of the compound of Example 2 further improved neurological function and this improvement was stable and long lasting (FIG. 13).

3. Third pMCAO Study

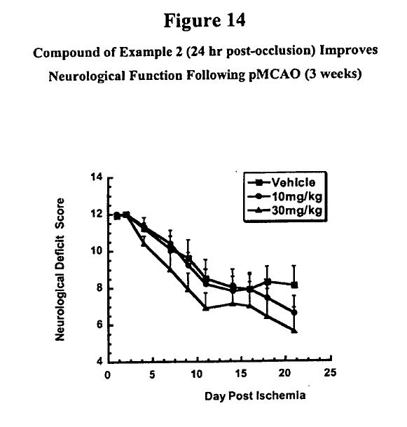

In the third study, the first dose of the compound of Example 2 is given 24 hours post-occlusion, followed by 4 additional doses, 24 hours apart (5 total doses). Neurological deficits were monitored over a 3-week period. 30 mg/kg of the compound of Example 2 significantly improved and accelerated neurological function when compared to vehicle treatment. 3 mg/kg and 10 mg/kg of the compound of Example 2 did not improve neurological function (FIG. 14).

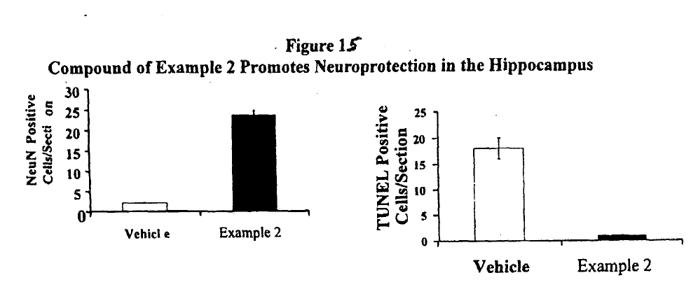

Brains from animals in the third study were collected and processed for neuronal, regenerative, and cell death markers. 30 mg/kg of the compound of Example 2 promotes neuroprotection (Neu-N positive cells) while reducing apoptotic cell death (TUNEL labeling) in the hippocampus (FIG. 15).

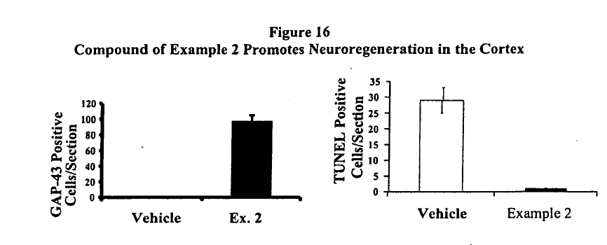

Similarly, 30 mg/kg of the compound of Example 2 promotes neuroregeneration (GAP-43 labeling) in the cortex (FIG. 16).

These data demonstrate that the compound of Example 2, which is a non-immunosuppressive immunophilin ligand, promotes neuronal survival and neurite outgrowth in cultured cortical neurons. The data further provides evidence of the utility of the compound of Example 2 for the treatment of ischemic stroke. Significantly, the compound of Example 2 is efficacious in two rat models of ischemic stroke and shows clinically relevant therapeutic windows of at least 24 hours. Moreover, the improvements in neurological function are stable and long lasting, up to 3 months post-occlusion.

All patents, patent publications, and other publications listed in this specification are incorporated herein by reference. While the invention has been described with reference to several embodiments, it will be appreciated that modifications can be made without departing from the spirit of the invention. Such modifications are intended to fall within the scope of the appended claims.

What is claimed is:

1. A method of treating a neurological disorder comprising administering to a subject in need thereof a compound of Formula I, and a pharmaceutically acceptable carrier, where the compound of Formula I has the structure: 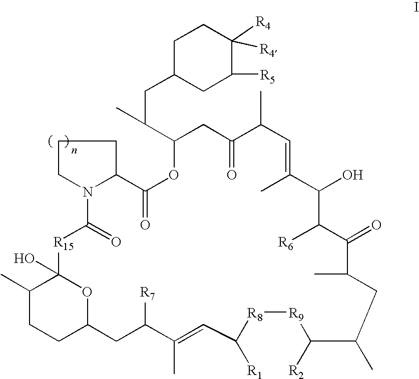
wherein:
R₁ and R₂ are different, independent groups and are selected from OR₃ and N(R_{3′})(R_{3″}); or
R₁ and R₂ are different, are connected through a single bond, and are selected from O and NR₃;
R₃, R_{3′}, and R_{3″}, are independently selected from H, C₁₋₆alkyl, substituted C₁₋₆alkyl, C₃₋₈cycloalkyl, substituted C₃₋₈cycloalkyl, C₆₋₂₀aryl, optionally substituted C₆₋₂₀aryl, heteroaryl, and substituted heteroaryl;
R₄ and R_{4′} are:
(a) independently selected from H, OH, O(C₁₋₆alkyl), O(substituted C₁₋₆alkyl), O(acyl), O(C₆₋₂₀aryl), O(substituted aryl), and halogen; or
(b) taken together to form a double bond to O;
R₅, R₆, and R₇ are independently selected H, OH, and OCH₃;
R₈ and R₉ are connected through a (i) single bond and are CH₂ or (ii) double bond and are CH;
R₁₅ is selected from C═O, CHOH, and CH₂; and
n is 1 or 2;
or a pharmaceutically acceptable salt thereof;
wherein said compound is administered to said subject at least 4 hours after the onset of said disorder or a symptom thereof.

2. A method of claim 1, wherein brain function is substantially restored to where it was before the onset of the neurological disorder.

3. A method of claim 1, wherein ischemia associated with the neurological disorder is reversed.

4. A method of claim 1, wherein the neurological disorder is a brain disorder selected from complications due to stroke, head trauma, spinal cord injury or traumatic brain injury.

5. The method of claim 1, wherein R₁ is O, R₂ is NR₃.

6. The method of claim 1, wherein R₁ is OR₃ and R₂ is N(R_{3′})(NR_{3″}).

7. The method of claim 1, wherein R₃, R_{3′} or R_{3″}, is a C₆₋₂₀aryl or optionally substituted C₆₋₂₀aryl.

8. The method of claim 7, wherein said C₆₋₂₀aryl or optionally substituted C₆₋₂₀aryl is of the structure: 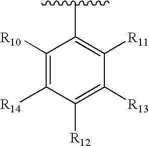
wherein:
R₁₀, R₁₁, R₁₂, R₁₃, and R₁₄ are independently selected from H, C₁₋₆alkyl, substituted C₁₋₆alkyl, C₆₋₂₀aryl, optionally substituted C₆₋₂₀aryl, heteroaryl, substituted heteroaryl, halogen, acyl, OH, O(C₁₋₆alkyl), O(substituted C₁₋₆alkyl), O(C₆₋₂₀aryl), O(substituted aryl), O(acyl), NH₂, NH(C₁₋₆alkyl), NH(substituted C₁₋₆alkyl), NH(C₆₋₂₀aryl), NH(substituted aryl), and NH(acyl).

9. The method of claim 1, wherein R₄ or R_{4′} is OH.

10. The method of claim 1, wherein R₄ or R_{4′} is O(acyl).

11. The method of claim 10, wherein said acyl is:

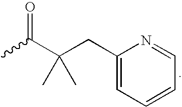

12. The method of any one of claims 1, wherein R₅, R₆, and R₇ are OCH₃.

13. The method of any one of claims 1, wherein n is 2.

14. The method of any one of claims 1, wherein R₁₅ is C═O.

15. The method of any one of claims 1, wherein R₁ and R₂ are connected through a single bond; R₁ is O; R₂ is NR₃; R₃ is phenyl; R₄ is OH; R₅, R₆ and R₇ are OCH₃; and R₈ and R₉ are HC═CH.

16. The method of any one of claims 1, wherein R₁ and R₂ are connected through a single bond; R₁ is O; R₂ is NR₃; R₃ is phenyl; R₄ is OH; R₅, R₆ and R₇ are OCH₃; and R₈ and R₉ are H₂C—CH₂.

17. The method of any one of claims 1, wherein R₁ and R₂ are connected through a single bond; R₁ is O; R₂ is NR₃; R₄ is OH; R₅, R₆ and R₇ are OCH₃; R₈ and R₉ are HC═CH or H₂C—CH₂; and R₃ is selected from

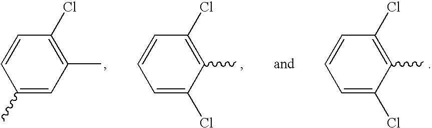

18. The method of any one of claims 1, wherein R₁ and R₂ are connected through a single bond; R₁ is O; R₂ is NR₃; R₃ is phenyl; R₅, R₆ and R₇ are OCH₃; R₈ and R₉ are HC═CH; and R₄ is

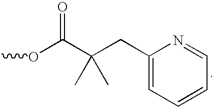

19. The method of any one of claims 1, wherein the compound of Formula I is selected from:

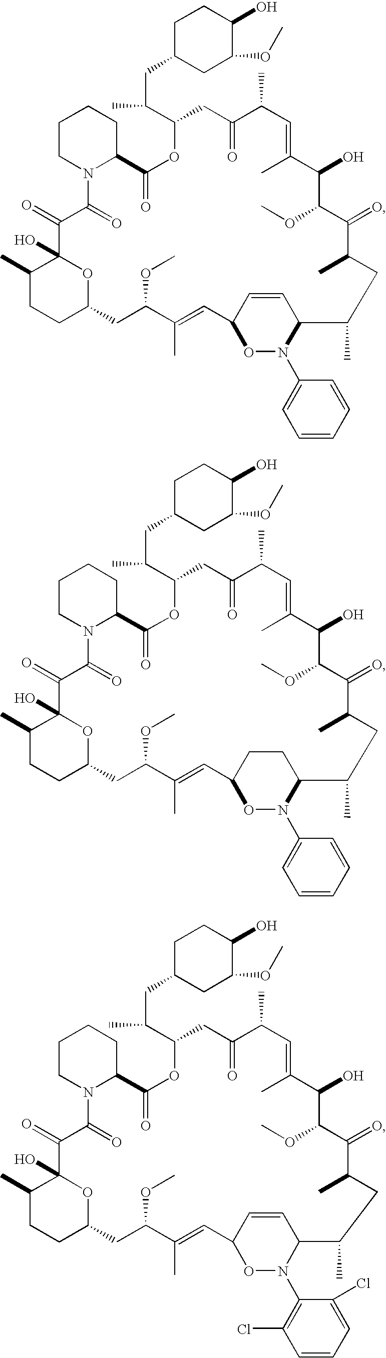

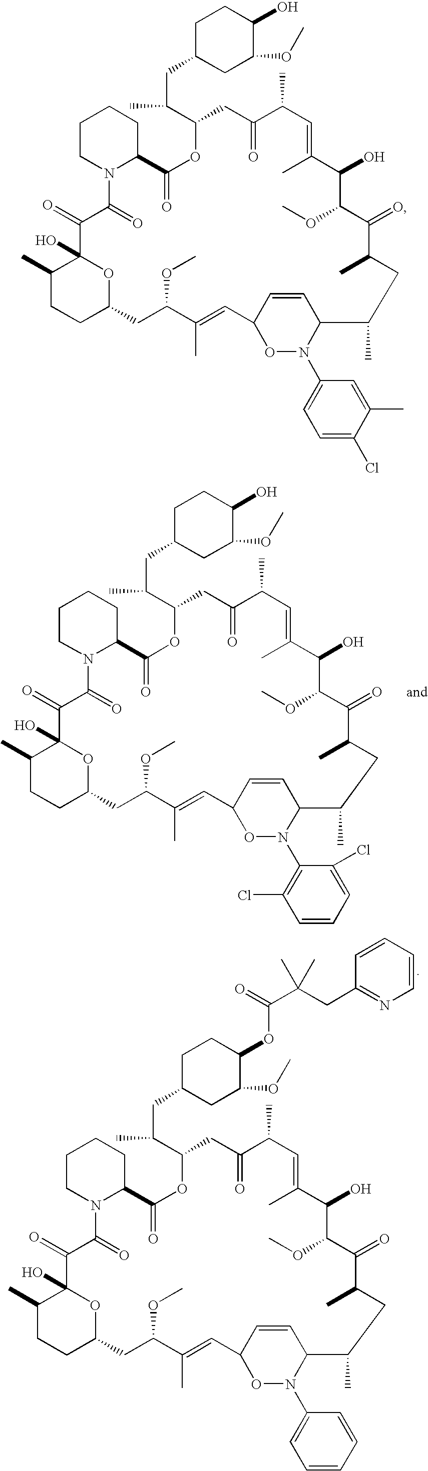

20. The method of any one of claims 1, wherein the compound of Formula I has the Formula Ia: 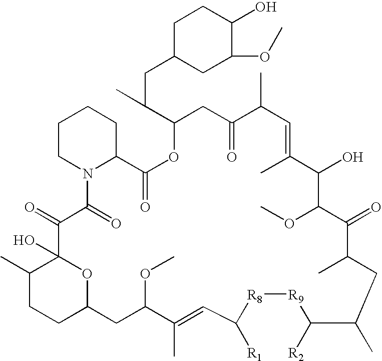
Ia
wherein:
R₁ and R₂ are different, independent groups and are selected from OH and N(R_{3′})(R_{3″}); or
R₁ and R₂ are different, are connected through a single bond, and are selected from O and NR₃;
R_{3′} and R_{3″} are independently selected from H, C₁₋₆alkyl, substituted C₁₋₆alkyl, C₃₋₈cycloalkyl, substituted C₃₋₈cycloalkyl, C₆₋₂₀aryl, optionally substituted C₆₋₂₀aryl, heteroaryl, and substituted heteroaryl;
R₈ and R₉ are connected through a (i) single bond and are CH₂ or (ii) a double bond and are CH; or a pharmaceutically acceptable salt thereof.

21. The method of claim 1, wherein the compound of Formula I has the Formula Ib: 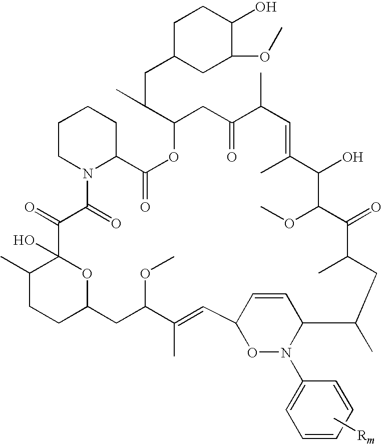
wherein:
R is independently selected from H, C₁₋₆alkyl, substituted C₁₋₆alkyl, C₆₋₂₀aryl, optionally substituted C₆₋₂₀aryl, heteroaryl, substituted heteroaryl, halogen, acyl, OH, O(C₁₋₆alkyl), O(substituted C₁₋₆alkyl), O(C₆₋₂₀aryl), O(substituted aryl), O(acyl), NH₂, NH(C₁₋₆alkyl), NH(substituted C₁₋₆alkyl), NH(C₆₋₂₀aryl), NH(substituted aryl), and NH(acyl); and
m is 1 to 5.

22. The method of claim 1, wherein said neurological disorder is selected from: venous cardiovascular thromboembolic disorders, thromboembolic disorders in the chambers of the heart, atherosclerosis, restenosis, peripheral arterial disease, coronary bypass grafting surgery, carotid artery disease, arteritis, ischemic heart disease, cardiac ischemia, ischemia, ischemic sudden death, transient ischemic attack, stroke, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, thrombosis, supraventricular arrhythmia, atrial arrhythmia, atrial flutter, and atrial fibrillation.

23. The method of claim 1, wherein the compound is administered to said subject at least 6 hours and can be at least 24 hours or longer after the onset of said disorder or symptom thereof.

24. The method of claim 1, wherein said compound of Formula I is prepared from norrapamycin, deoxorapamycin, or desmethylrapamycin starting material.

25. The method of claim 24 wherein the deoxorapamycin is

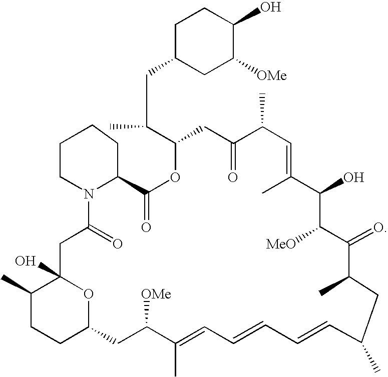

26. The method of claim 24 wherein the desmethylrapamycin is selected from [chemical structure]
